(19) United States
(12) Patent Application Publication
(10) Pub. No.: US 2024/0215864 A1
(43) Pub. Date: Jul. 4, 2024

(54) SYSTEMS AND METHODS FOR NEUROMUSCULAR FEEDBACK SMART WEARABLES TO OPTIMIZE MOVEMENT HEALTH AND PERFORMANCE VIA JOINT STABILITY

(71) Applicant: MINDFULL SOLUTIONS INC., Calgary (CA)

(72) Inventor: Nigel A. JOSEPH, Calgary (CA)

(21) Appl. No.: 18/558,125

(22) PCT Filed: Apr. 29, 2022
(86) PCT No.: PCT/CA2022/050666

§ 371 Date: Oct. 30, 2023

Related U.S. Application Data
(60) Provisional application No. 63/182,246, filed on Apr. 30, 2021.

(51) Int. Cl. A61B 5/11; A61B 5/00

(52) U.S. Cl. CPC A61B 5/1114; A61B 5/1116; A61B 5/4538; A61B 5/486; A61B 2562/0219

(57) ABSTRACT

The described technology generally relates to wearable devices, smart apparel, systems and methods for movement wellbeing, and performance through haptic feedback. One or more wearable devices (e.g., standalone or incorporated into apparel, accessories, or other wearable items) may promote neuromuscular feedback for joint centration often referred as joint stability. Joint centration or joint stability combines the functioning cartilage, ligaments, fascia, tendons, and muscles that allow joints to stay secure as forces transfer through them during movement with minimal stress on the passive soft structures. In some embodiments, there may be an electronic control unit with at least one sensor to detect user's joint movement, such as movement on a three-axis position or joint speed, and acceleration. Depending on the movement of the users, the electronic control may communicate joint movement to a user and provide cues for joint stability, joint movement range, muscle memory, and body position awareness.

This application is a PCT patent application claiming priority to and the benefit of U.S. Provisional Application No. 63/182,246, filed Apr. 30, 2021, such patent application and any priority case hereby incorporated herein by reference in its entirety.

### FIELD OF THE INVENTION

Embodiments of the application generally relate to the field of movement wellbeing neuromuscular feedback apparatuses and methods of using the same. More specifically, embodiments, may relate to systems and methods for neuromuscular feedback smart wearables, perhaps to increase tensegrity of muscles and fascial systems and joint centration, joint stability, or even use in movement feedback training, movement feedback rehabilitation, performance measurement, ergonomic optimization, or the like.

### BACKGROUND OF THE INVENTION

Healthy and successful movement composes of joint centration or joint stability as the body chooses the best position and muscle sequence to match the environment or situation. Human joints are made up of an intertwined soft tissue structure. Human joints are intertwined soft tissue structures. The soft tissue may have some laxity, and as the laxity increases, so may the diversity of human movement, making joint stability even more critical. Joint centration or joint stability combines the functioning cartilage, ligaments, fascia, tendons, and muscles that allow joints to stay stable as forces transfer through them during movement with minimal stress on the passive soft structures (Kobesova, Safarova, & Kolar, 2016). If one component falters, the other components take more responsibility for keeping the joint stable. Slight movement (range of motion or angular velocity) and/or fluid or smoother stop/start (acceleration) causes less strain on the joint. Conversely, large angular velocity and abrupt acceleration may cause a greater amount of force to be applied to the joint. Understanding when and how much joint stability a person needs to transfer forces safely and effectively for movement is a complex art and science. If people do not move in a variety of ways, we disconnect from how much stability is required to transfer forces through the joint. Also known as too much or too little muscle and fascial tensegrity (Bordoni et al., 2019). Tensegrity refers to a structure that correctly manages mechanical tensions and transmits them throughout the body. Inappropriate muscles and fascial tensegrity may become detrimental as speed and complexity of our movement (running vs. walking) increases. As higher forces pass through the joint, increasing the likelihood of aggressive wobbles and sways it may increase the likelihood of both traditional understood joint instability and opposite joint inflexibility. Over time, if muscle or fascial tension is insufficient, then significant wobbles, inflexibility, or sways may cause harm to the joint structures, lessening the joint function and altering body awareness, movement patterns, and/or muscle memory.

Joint health may be the second largest reason a person sees a doctor and can be a significant challenge to international health systems as many conventional solutions only treat the symptoms of pain instead of addressing what can often be a possible root cause, that being weak stabilizers of overdeveloped primary movement muscles. Movement injuries, such as but not limited to hamstring strains, non-contact ACL tears, and shoulder dislocations, are usually the result of joint instability or unequal development of the supporting musculature that may alter natural kinetic paths. Underdeveloped and unequal supporting musculature and joint instability can result in an increase in injuries and a decrease in movement performance. One common problem among people with joint instability is larger peak joint angles during activities—for example, but not limited to, pelvic tilt as but one example. Moreover, increasing speed while performing dynamic movements may cause greater peak joint angular velocity and acceleration, resulting in a greater amount of joint instability, which may lead to acute or chronic pain and injuries. Conventional methods of increasing joint stability may utilize increasing neuromuscular activity through a passive stimulus or active neuromuscular feedback. Kinesiology tape and compression fabric may have been found to aid movement learning: however, kinesiology tape may cause skin irritations and can be difficult to apply without professional assistance. Compression fabric (e.g., medical-grade and commercial grade) can have a “Goldie Locks” complex. Medical grade compression can be too constrictive, while commercial compression may not be stiff enough to achieve neuromuscular feedback.

Most recently, electronic devices have grown in popularity and can help users self-discover movement health. One advantage may be that it may allow a more significant portion of the population to interact with their movement wellbeing easily. However, these products do not usually let users safely share metrics and readings with their clinician, practitioner, coach or trainer for oversight. Furthermore, there remains a desire for a consumer device that can allow for user and clinician integration as telehealth becomes a more integral part of health-related clinical visits. Another potential area where current fitness trackers and data measurement systems fall short of consumer expectations is that most rely on infrared sensors, which do not work effectively with pigmented skin and adipose tissue which may cause many issues for athletes and users with darker skin pigmentation.

Traditionally, when studying and measuring movement, we measure the articulating segments that make up movement, mostly focusing on triangulation of rigid masses and prime mover muscles. In doing so, it makes the math simpler to quantify overall movement performance, health, and wellbeing. It also assumes our joints are ridged masses all the time. This assumption is limiting as the structures that make up the joint are composed mostly of soft tissue. An example is the Lumo Run™ sensor device. Lumo Run™ may include an accelerometer and a microprocessor that analyzes directional movement data from the accelerometer and assesses the user's posture in that movement state (i.e., a posture quality of the user). The feedback may be provided in real-time through an actuator that provides sensory activity (e.g., vibration or sound) and/or through an avatar within a corresponding application with which the sensor device communicates. Lumo Run's™ U.S. Pat. Pub. No. 2013/0207889, hereby incorporated by reference herein, relates that posture may be detected and may send feedback that helps users find a good posture, including a neutral spine in a seated, lying, standing, and moving position. Conversely, users' core musculature and boney structures come in all shapes and sizes. The boney structure and musculature of an Olympic 100 meter sprinter may be vastly different from a person who lives with multiple sclerosis or a woman in her third trimester of pregnancy: nevertheless, their success in finding movement wellbeing may be the same, increasing joint stability, particularly as you increase intensity and speed of their movements.

Many of the same movement wellbeing devices may cue ideal movement using different methods, including sound, vibration, lights and images, and videos. Also, many devices may rely on external devices to aid in cueing feedback, such as a smartphone, smartwatch, tablet, or computer giving the user real-time data. However, focusing on external devices may negatively impact skill acquisition and performance like movement education. The majority of external devices are designed for multifunctional purposes, allowing users to do multiple tasks simultaneously, including entertainment and communication. One potential result and draw back may be that the user is not fully present in learning a new skill, which potentially reinforces undesired movement habits.

Additionally, humans are widely known for being visual creatures, relying on visual feedback for learning and adapting to our environment. External devices are great for giving visual feedback. However, when executing movement, environmental visual cues may be required for success, such as but not limited to hitting a tennis ball, lifting a box, or stepping onto a curb. WearableX's™ PCT Pat. Pub. No. WO 2017/120367, hereby incorporated by reference herein, may provide a smart garment that connects to an external device such as the user's smartphone to facilitate the execution of a physical activity. Moreover, the user can select specific activities, movements and/or poses on their external device and provides real-time feedback on execution. WearableX™ may be a smart garment that gathers information about a wearer of the smart garment undertaking physical activity, such as but not limited to yoga, with one or more actuators to provide haptic feedback to the user.

### SUMMARY OF THE INVENTION

In general, the present application may involve both devices and methods in a variety of embodiments, to achieve optimized joint function and stability via neuromuscular, perhaps feedback smart wearables. In one embodiment, implementations described herein may address a system for one or more wearable devices (e.g., standalone or incorporated into apparel, accessories, or other items a person may wear such as but not limited to structural tapes, adherable movement sensors, or the like) that can promote neuromuscular feedback. In embodiments, the device may use feedback to guide joint stability thereby possibly increasing the user's awareness of their body, body position, movement patterns, and/or muscle memory.

Human joints are made up of an intertwined soft tissue structure. The soft tissue is intended to have some laxity as to achieve the diversity of human movements humans are able to accomplish safely. Small movement (range of motion or angular velocity) and/or fluid or smooth or acceleration stop/start may cause less strain on a joint. Conversely, large angular velocity and abrupt acceleration can cause a greater amount of forces to be applied to the joint. Understanding when and how much joint stability a person needs to transfer forces safely and effectively for movement is a complex art and science. Stability in this sense is meant to encompass conditions where forces on and activities by the joint can occur without detriment. This can occur sustainably in the sense that long term performance is not degraded as a result of that movement. To determine the stability that is required to transfer forces through the joint, it may be imperative that humans move in a variety of ways so that the forces can be measured. Without gathering joint stability measurements, it may be difficult to estimate a safe and sustainable human joint movement. Based on differing joint anatomies from human-to-human individual joint measurements may be required to be taken to measure and train humans for sustainable and safe joint movements. The difficulty in estimating safe and sustainable human joint movements may become more difficult as we add speed and complexity to human movement (running at high speeds-higher complexity versus sitting in a chair-lower complexity) as a higher amount of forces pass through the joint increasing the likelihood of joint instability, aggressive wobbles, and sways. Over time, if muscle engagement does not control the joint stability, then the joint instability and aggressive sways may cause harm to the soft tissue, increasing the laxity of the joint and impacting joint stability and overall movement. This may lead to an increase in the probability for injury, increased joint discomfort, or a decrease in athletic performance. Measuring joint stability may be accomplished by measuring the angular velocity, range of motion, acceleration on, or rotation of the joint that may be caused by muscle tension surrounding the joint. Further, aspects that can be used in making a stability or even a range determination (which could be an amount, upper and lower values, a threshold, or the like) can be one or more of items such as: joint optimal movement aspects (such as can be understood from a proper or optimal joint were that user to have such a thing), user baseline aspects (such as from a first or prior use of the device), consideration of at least one contemporaneously sensed joint movement (such as the then experienced acceleration, velocity, or the like), muscle activation (both existence and amount as either too little or too much can be undesirable), joint muscle activation (such as the muscles at, in, or adjacent to the joint in question), and even a separate muscle area such as core muscle activation (the core being abdomen, etc.) and other factors. These can be used to determine a preferred range, to evaluate stability, or otherwise.

Haptic feedback is something many do to teach prescribed movements from either a library of movement or a mathematical algorithm for idealized movement. However, ideal is not what the movement looks like, but how functional the movement is from a joint health perspective. It may be more advantageous to measure and to be alert to actual human movement or occurrences rather than an ideal model generated from a computer model.

Measuring joint segments is one biomechanical perspective on movement. However, an assumption of measuring two segments above and below the joint may be detrimental to sustainable or perhaps stable joint movement because in measuring a joint segment, what happens at the segments can determine what happens at the joint. However, measuring at the joint segment may not always be the best determination of joint stability because of passive joint stiffness. Passive joint stiffness is the ability to control the joint by engaging the muscles around the joint to control joint acceleration in a peak joint range of motion.

A large amount of human movement and stability control may be controlled or impacted by core muscle stability and the joint movement of the low back in the spine and sacroiliac joint. To train a human for sustainable joint movements by creating a stable joint haptic feedback to limb muscles and low back region (L4, L5, S1 joints as a non-limiting example) may be beneficial. In some embodiments, the user feedback signal, including but not limited to haptic feedback, may be applied to the lower back, and in others, it may be applied to a limb joint such as but not limited to the knees, ankle, or hips.

In embodiments, electric current generated by a chargeable power source integrated into a wearable device may power a sensor, such as but not limited to an inertial sensor, accelerometer, movement sensor, position based sensor, velocity sensor, jerk sensor, micro electro mechanical system sensor, or the like which may detect user metrics such as but not limited to motion, movement, position, or the like. A user's joint movement, joint acceleration, and/or joint angular velocity may be detected by a device where an electric current may be generated and transmitted through a transmission medium, such as conductive filament, wire, wirelessly through Bluetooth, other wireless connectivity media, or the like perhaps to at least one actuator located either in the device, apparel, accessories, or other items a person may wear. The device/smart apparel function may allow users to connect with their body on a deeper, more intentional level to create an image and feeling of the required muscle activation instead of relying on an external device that may alter the desired joint stability and distract the user to focus on subjective movement feedback. Subsequently, inertial sensor detection or other information data may be stored and transmitted through a transmission medium to an external device, platform, or computer-readable storage medium. This allows parties, such as but not limited to an athlete and trainer or the like, to utilize data similar to how racecar drivers and engineers may use vehicle data to improve driving performance. This may improve the performance of the athlete and the movement wellbeing.

The present application may be captured in different embodiments, including but not limited to smart active wear, musculoskeletal support structures, structural tape, adherable movement sensors, or the like. Naturally, further objects, goals and embodiments, of the application are disclosed throughout other areas of the specification, claims, and drawings.

### BRIEF DESCRIPTION OF THE DRAWINGS

The implementations described herein may be better understood with reference to the following drawings and descriptions. The components illustrated in the figures are not to scale, and therefore the figures may illustrate the operating principles of the various embodiments, of innovation. Like reference numbers designate corresponding components and functions throughout the described technology.

FIG. 1 illustrates an exemplary embodiment, of joint stability or centration of motion according to some embodiments.

FIG. 2 illustrates an exemplary embodiment, of a device and/or clothing that detects joint movement according to some embodiments.

FIG. 3 illustrates exemplary operations for a joint stability system, including the electronic control unit according to some embodiments.

FIG. 4 illustrates an exemplary embodiment of a device and or clothing that cues for muscle and fascial tensegrity according to some embodiments.

FIG. 5 illustrates exemplary operations of a joint stability system, including the electronic control unit according to some embodiments.

FIG. 6 is an exemplary embodiment of joint stability acceleration and angular velocity data versus time.

FIG. 7 is an exemplary embodiment, of a method of measuring joint stability.

FIG. 8 is an exemplary embodiment, of a method of providing joint range feedback.

FIG. 9 is an exemplary embodiment, of a method of measuring inter-joint stability.

### BRIEF DESCRIPTION OF THE PREFERRED EMBODIMENTS

It should be understood that embodiments, can include a variety of aspects, which may be combined in different ways. The following descriptions are provided to list elements, and describe some of the embodiments, of the application. These elements are listed with initial embodiments: however, it should be understood that they may be combined in any manner and in any number to create additional embodiments. The variously described examples and preferred embodiments, should not be construed to limit the embodiments of the application to only the explicitly described systems, techniques, and applications. The specific embodiment, or embodiments, shown are examples only. The specification should be understood and is intended as supporting broad claims as well as each embodiment, and even claims where other embodiments, may be excluded. Importantly, disclosure of merely exemplary embodiments, is not meant to limit the breadth of other more encompassing claims that may be made where such may be only one of several methods or embodiments, which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

In some embodiments, a neuromuscular feedback smart wearable system or device (e.g., standalone or incorporated into apparel, accessories, or other items a person may wear) may promote neuromuscular feedback in various implementations. The basic components that make up a device that promotes neuromuscular feedback may include one or more of the following: power source, an inertial sensor, a central or separate processing unit, actuator, protective casing, or the like.

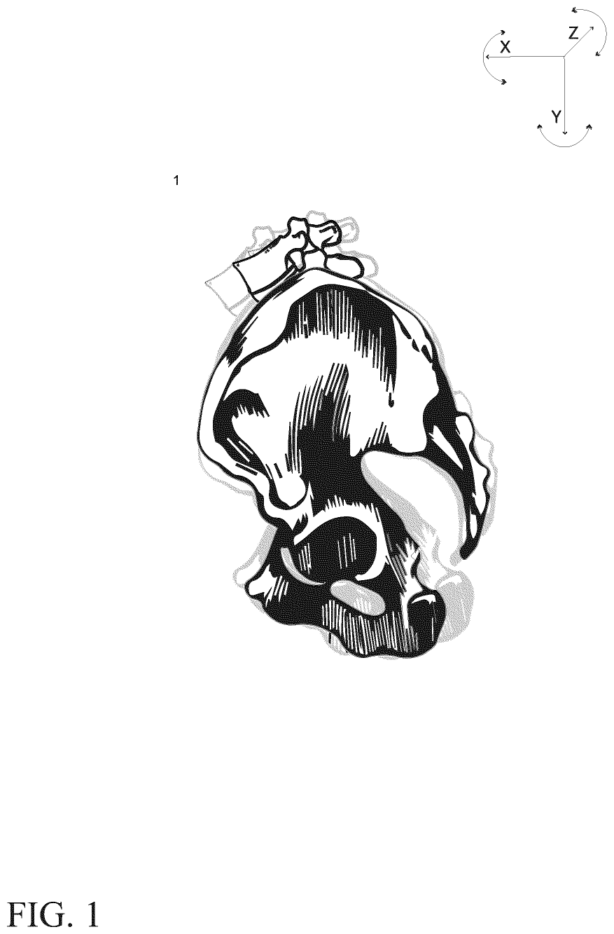

FIG. 1 illustrates an exemplary embodiment, of joint stability or centration of motion according to some embodiments. A sustainable joint movement may use the pelvis structure in the anterior and posterior pelvic tilt as an example. Sustainability with relation to a joint movement may be related to what may be achieved over the period of a human's life without causing pain, discomfort or injury. Sustainable joint movement may be present in other joints structures, including but not limited to, wrist, elbow, shoulder, collar, ankle, knee, or any other joints perhaps understood by consideration of one or multiple dimensions, such as three dimensions. These dimensions may be most easily understood with the three-axis position (X, Y, and Z) or X, Y, Z coordinate plane.

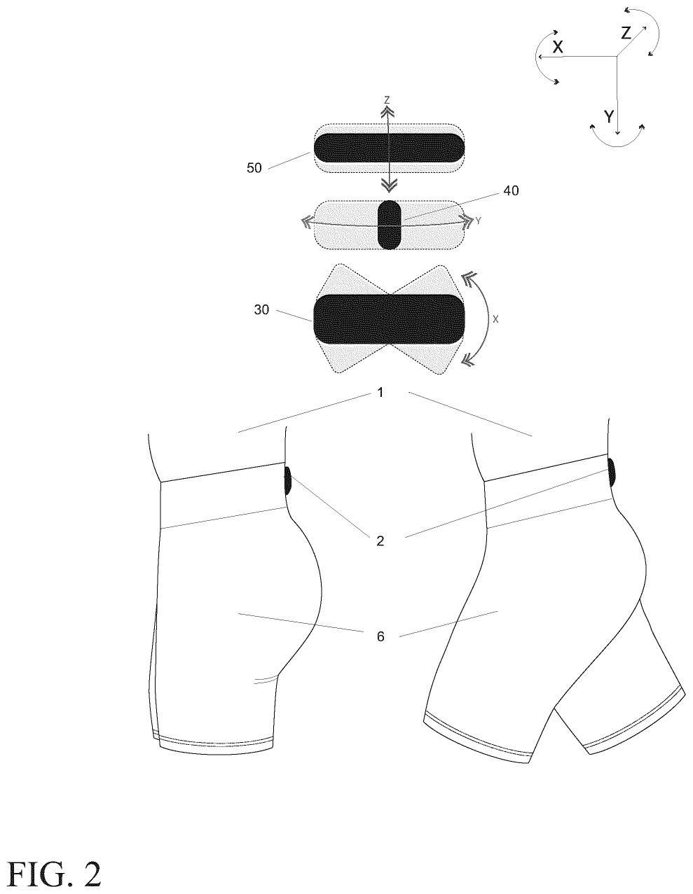

FIG. 2 illustrates an exemplary embodiment, of a device and/or clothing that can detect joint movement. A joint movement may use the pelvis structure and anterior and posterior range of motion and acceleration as a factor in joint stability. In this sense acceleration is meant in the sense of the effect from a muscular or external force on a joint. A neuromuscular feedback smart wearable system may be but is not limited to an article of clothing, such as depicted in FIG. 2 or any other wearable device. The neuromuscular feedback smart wearable system (2) may be shown detecting joint movement along, but not limited to, a three-axis position (X, Y, and Z) during different activities, including but not limited to, standing, walking, or the like. When the user (1) moves, the device or clothing (2) may notify the user (1) via haptic feedback during a binary condition. For example, a user sprinting 300 meters in an interval workout, a first condition may be when the user (1) is outside the sustainable anterior/posterior pelvic tilt and high rates of acceleration, the neuromuscular feedback smart wearable system (2) may send immediate feedback, such as but not limited to a pulse haptic vibration until sustainable anterior/posterior pelvic tilt or slowing the rate of acceleration is achieved. A second condition may be when the user (1) is within a calculated or determined sustainable joint stability, the device or clothing (2) may send randomized interval feedback, such as but not limited to a slow wave haptic vibration once every four to thirty seconds.

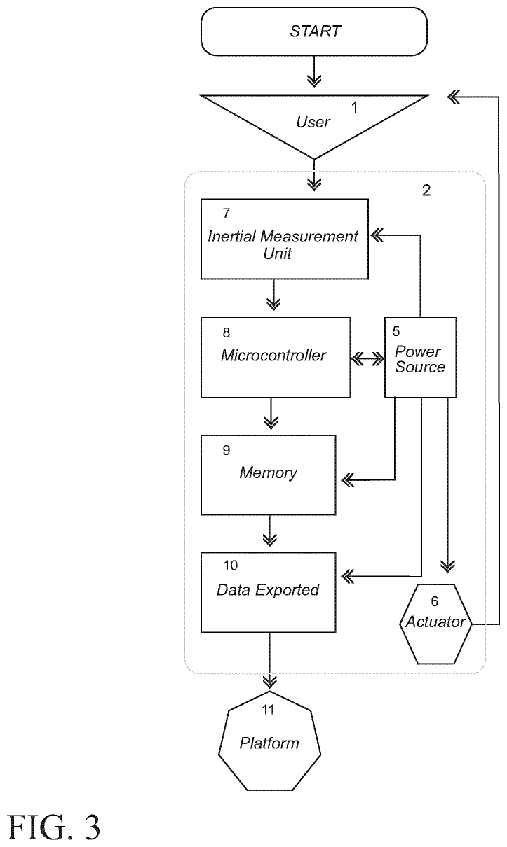

FIG. 3 illustrates exemplary operations for a joint stability system, including the electronic control or analysis unit in some embodiments. A neuromuscular feedback smart wearable system may be similar to the example shown in FIG. 2, but FIG. 3 displays it as an example of a simplified program or process flow diagram. The depiction of neuromuscular feedback smart wearable system (2) may include a power supply (5), a joint movement sensor. Perhaps established as an inertial measurement system (6), a microcontroller (8), an indication—perhaps established as an internal actuator (7), a computer readable memory component (9), a transmitter (10), and an external platform (11), such as but not limited to a cellular phone or computer application, or the like.

In embodiments, the neuromuscular feedback smart wearable system (2) and the power supply or more generally mobile power system (5) may allow the system to function by allowing some movement sensing, perhaps such as an inertial measurement system (7) to detect joint movement and facilitate determining stability, such as in the pelvis. The inertial measurement system (7) may then transmit data, by but not limited to a wireless communication or through a wired electrical signal, to a microcontroller (8), which may have multiple operations. A microcontroller (8) may be configured and arranged to determine joint range information, that is any information indicative of or derived from some aspect of a joint range (e.g., the actual range, its upper bound, its lower bound, its extent, etc.). This joint range information may be a determined amount of range or values for a range that is deemed preferred for the activity, joint, and/or user involved, the conditions experienced, or in general. This may determine sustainable joint movement and send a signal to an internal actuator (6) to operate based on the movement of a user. An example of a pattern that may be utilized in embodiments, may include an interval to address core instability. In the neuromuscular feedback smart wearable system (2), a microcontroller (8) may communicate with an internal actuator (6) to operate, perhaps achieving an indication or having an indicator that may be with respect to information that may be used then or later. The indicator may merely export data or may also send feedback to the user (1) if the user is detected to be outside the identified sustainable tilt angle, range, is unstable in a joint, or is suboptimal in an acceleration rate based on movement and activity. And further, the indicator may be a preferred joint range information-based indicator in that any information that is based on any component of a range determination can be indicated to a system, a central processor, or even a user. In other embodiments, a microcontroller (8) may either internally store or send movement data to a computer readable memory component (9) and transmit the data to an external platform (11).

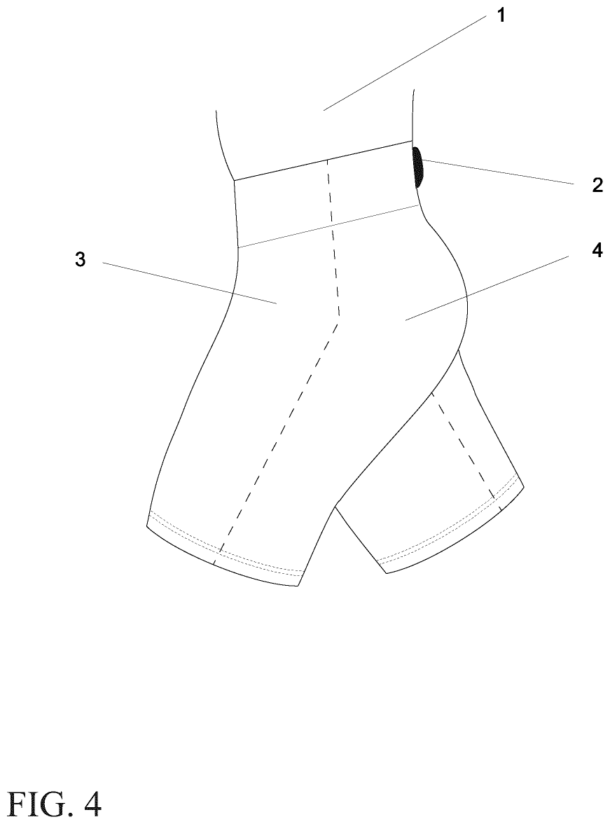

FIG. 4 illustrates an exemplary embodiment of a device and or clothing that cues for muscle and fascial tensegrity according to some embodiments. Muscle activation may be cued through walking patterns as an example. This embodiment of the neuromuscular feedback smart wearable system may be similar to FIG. 2, but FIG. 4 displays an example cueing joint stability instead of merely detecting joint movement in the x (30), y (40), and z (50) direction. In the embodiments in FIG. 4, the neuromuscular feedback smart wearable system (401) may be detecting joint conditions such as but not limited to joint movement, speed, acceleration, and/or position. When the user (1) moves, the neuromuscular feedback smart wearable system (2) may notify neuromuscular feedback sensors (6 in FIG. 3) to operate, possibly sending haptic feedback to identified movement muscle groups, for instance, but not limited to anterior (3) and posterior (4) chain groups of upper leg and buttock (5).

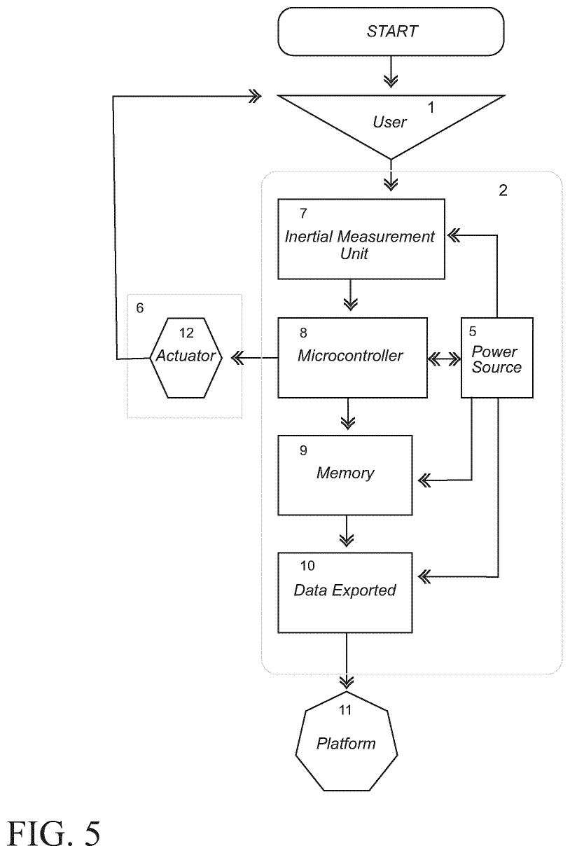

FIG. 5 illustrates exemplary operations of one joint stability system, including the electronic control unit according to some embodiments. A neuromuscular feedback smart wearable system (2) may include a wearable device or clothing with similar components to FIG. 3, but FIG. 5 displays a simplified program or process flow used in a muscle memory system. The neuromuscular feedback smart wearable system's main features may still be present: a power supply (5), a movement sensor such as an inertial measurement unit (7), a microcontroller (8), a computer readable memory component (9), a transmitter (10), and external platform (11), or the like.

A neuromuscular feedback smart wearable system (501) may include a power source or supply (5) which may power the system allowing an inertial measurement system (7) to measure joint movement, such as pelvic tilt and rate of acceleration, including but not limited to joint position and even joint speed. A microcontroller (8), which may have multiple functions, may then direct the system to record the data transmitted from an inertial measurement system. In embodiments, the neuromuscular feedback smart wearable system (2) may provide a microcontroller (8) which may communicate with an actuator (12) as a part of a muscle chain group (3 or 4). An actuator may be embedded into apparel to operate almost simultaneously sending feedback to the user quickly cueing ideal sequential muscle firing patterns or the like.

In some embodiments, a microcontroller (8) may either internally store or even send movement data or determinations to a computer readable memory component (9) and transmit such movement data to an external platform. In the neuromuscular feedback smart wearable system (2), a microcontroller (8) may communicate with a computer readable memory component (9) to store data which may permit a transmitter (10) to transmit data through wired or wireless modalities to an external platform (11) such as, but not limited to a cloud network.

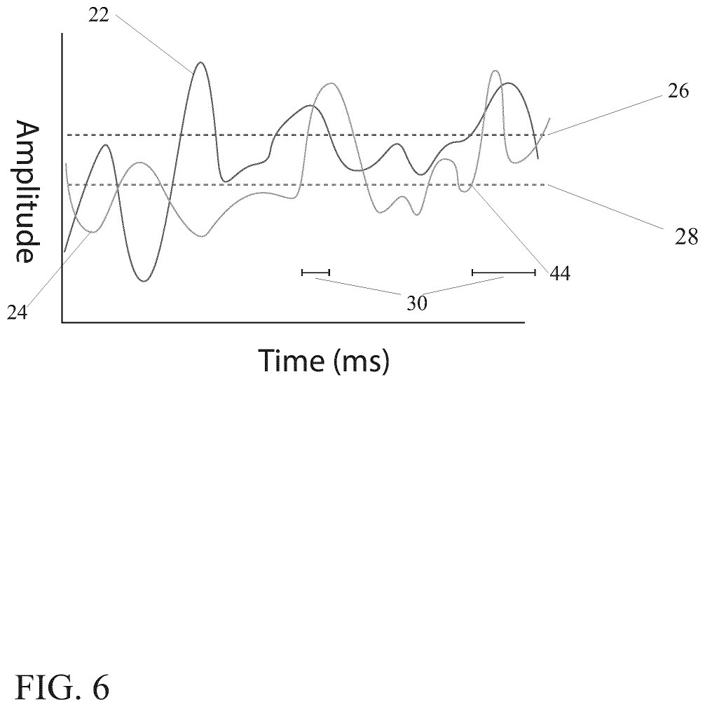

FIG. 6 is an exemplary embodiment of joint stability type movement, acceleration, and angular velocity data versus time. In some embodiments both joint acceleration and joint angular velocity may be used to determine and calculate joint stability. Joint stability data may show joint acceleration (22), joint angular velocity (24) (range of motion), acceleration threshold (26), angular velocity in real-threshold (28), potential time spent of joint instability (30), and haptic feedback trigger (44) when two or more threshold levels are exceeded.

In some embodiments, a joint stability measurement system (2), may include but is not limited to a mobile power system (5), a joint stability sensor (7), a microcontroller (8) configured and arranged to compute joint range as a function of joint acceleration and joint angular velocity in real-time, and a sustainable position joint range algorithm.

A mobile power system (5) may include a battery, a capacitor, a piezo-electric power generator, or the like. A mobile power system, that is one that can make with the user or such, may be helpful to provide adequate range of motion for a user during certain athletic movements such as running, walking, and/or jumping. A piezo-electric power generator may produce power upon oscillation of piezo-electric components, which may be beneficial to power a joint range measurement system in an active state.

In some embodiments, a joint stability sensor (7) may include but is not limited to an accelerometer, inertial measurement unit, or the like. In some embodiments, the accelerometer type may be of the piezoresistive, capacitive, or the like accelerometer. In some embodiments, an inertial measurement unit may be of the gyroscopic, magnetic, or the like type of inertial measurement unit. An inertial measurement unit may also include but is not limited to including accelerometers. In some embodiments, a joint-movement sensor may be that of an inertial sensor, inertial measurement unit or system, a gyroscope, an accelerometer, a combination of the aforementioned elements, or the like. An inertial sensor may be beneficial in easily sensing joint rotation and acceleration.

In some embodiments, a microcontroller configured and arranged to compute joint range as a function of joint acceleration and/or joint angular velocity in real-time (such as when in the moment of the activity) may be beneficial. Thus, there can be a real-time joint range processor, a real-time joint stability processor, and the like. Computing joint range as a function of joint acceleration and/or joint angular velocity in real-time, or close to real-time (such including in milliseconds, seconds, or coordinated to be accomplished during the specific activity, operation, or movement action involved) may be beneficial in providing optimal joint angle, position, or the like. Real-time, or close to real-time measurement and calculation may be important to measure a user's percentage of time in a situation, such as in unstable movement versus a sustainable movement (or a stable movement).

In some embodiments, the joint stability sensor that is an accelerometer, inertial measurement unit, or the like may be configured to measure non-optimal joint movement (not as an idealized, perhaps perfect joint would or should operate), lateral aspects (joint acceleration, lateral joint movement, core muscle stability, joint stability) as a function of supporting muscle tension, or the like. In some embodiments, it may be beneficial to take joint acceleration measurements at different types of joints or joint segments. In some embodiments, these joints may be but are not limited to a sacroiliac joint, a ball and socket joint a hinge joint, a condyloid joint, a pivot joint, a gliding joint, a saddle joint, or the like. As an example, measuring joint acceleration over the sacroiliac joint may provide the required data to predict sustainable movement for a user's back health.

In some embodiments, it may be beneficial to include a transmitter that may be used to transmit joint acceleration data to an external application or internal microcontroller. In some embodiments, the external application may be a cellular phone application, a computer application, a cloud computing system, or the like. Data may be processed by an external application or by a microcontroller that in some embodiments, may run a sustainable position joint range algorithm. In some embodiments, a sustainable position movement joint range processor or algorithm may be used to alert a user through haptic feedback of unstable joint movement.

In some embodiments, in order to gather joint acceleration data, it may be beneficial to contain joint stability processor or perhaps a joint stability measurement system in a wearable containment system. In some embodiments, a user may don a wearable containment system so the angular velocity sensing is in close proximity to the joint being measured. In some embodiments, a wearable containment system may be in the form of a compression pant, such as athletic tights, leggings: a compression sleeve, such as an arm sleeve, leg sleeve, calf sleeve, ankle sleeve, sock, or the like: a user-adjustable belt, a compression belt, or the like: or a compression shirt, such as a long sleeve, short sleeve, or tank top: a compression short: a compression underwear, in the form of briefs, trunks, boxer briefs, bikini, thong, or the like: a compression socks: a compression gloves, and the like. This may include a processor, perhaps such as a stability processor, and other processors or processor capability may be provided on a back end (in the cloud, on a smart phone, or otherwise).

In some embodiments, of devices, perhaps of a joint stability measurement system, it may be beneficial to further include a user feedback signal. In some embodiments, a user feedback signal may be a haptic feedback signal. Haptic feedback may also be known as kinesthetic communication or three-dimensional touch, and may refer to any technology that can create an experience of touch by applying forces, vibrations, or motions to the user. The haptic feedback may also be the use of advanced vibration patterns and waveforms to convey information to or to alert a user. In some embodiments, a haptic feedback signal may be an actuator configured and arranged to vibrate. In some embodiments, an actuator may be a small DC motor, a piezo-electric actuator, or the like. In some embodiments, an actuator may be configured and arranged to warm the skin of a user. An actuator configured and arranged to warm the skin may be a resistor or resistive actuator that may use electric energy to warm an area of the user's skin in contact with the joint stability measurement system. In other embodiments, a user feedback signal may be a visual indication. In some embodiments, a visual indication may be at least one LED (light emitting diode). In some embodiments, a singular LED may be used and, in some embodiments, an array of LEDs may be used. In some embodiments, a user feedback signal may be an external application notification. This external application notification may be broadcast to a phone application, computer application, or the like such can react or act appropriately.

In some embodiments, a sustainable position joint movement range processor may be a real-time joint movement range processor or even a sustainable position joint movement range processor that calculates the time between or of unstable and stable joint position, or the like. An unstable and stable joint may be different for different individuals. A stable joint may be one that does not have a high (above 60 percent) risk chance of injury whereas an unstable joint may be one that has a high risk of injury.

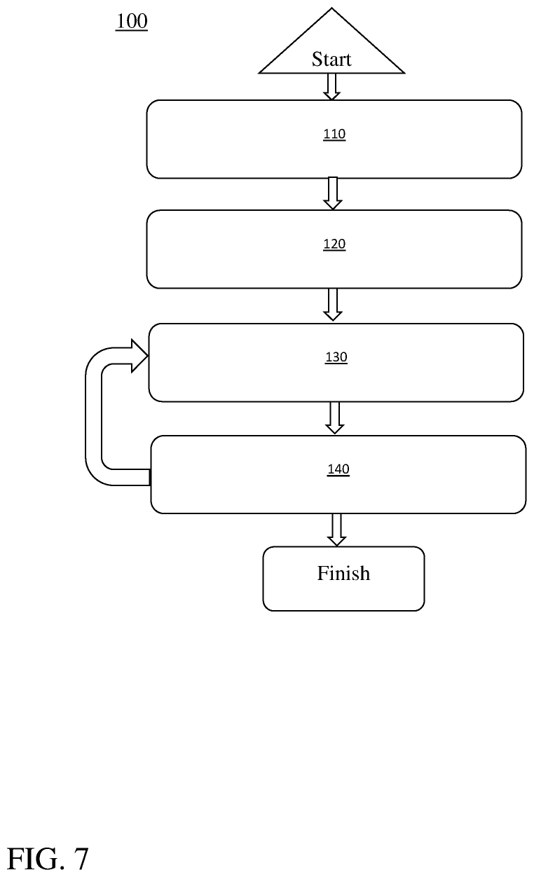

FIG. 7 is an exemplary embodiment of a method of measuring joint stability. In some embodiments, it may be beneficial to include a method of measuring joint stability (100). In some embodiments, a method of measuring joint stability may be accomplished by portably powering a joint movement sensor or a joint acceleration measurement system (110), providing at least one joint movement stability sensor (120), sensing joint movement or acceleration with at least one joint movement or stability sensor (130), calculating joint stability or range as a function of joint acceleration and joint angular velocity (140), and the like.

In some embodiments, portably powering a joint acceleration measurement system (100) may be accomplished by portably powering with a battery, a capacitor, a piezo electric power generator, or the like. A piezo electric power generator may be a series of piezo electric elements that upon introduction of a vibration, movement, or the like generates electricity.

In some embodiments, the step of providing at least one joint movement sensor (110) may be accomplished by providing an accelerometer, either a piezoresistive accelerometer, a capacitive accelerometer, or the like. In other embodiments, providing at least one joint movement sensor (110) may be accomplished by providing an inertial measurement unit. In some embodiments, providing an inertial measurement unit may be accomplished by providing a gyroscopic inertial measurement unit, a magnetic inertial measurement unit, an accelerometer inertial measurement unit, or the like.

In some embodiments, sensing joint acceleration with at least one joint movement sensor may be accomplished by sensing lateral joint acceleration, sensing lateral joint movement, sensing core muscle stability, sensing joint acceleration and perhaps joint angular movement or even velocity in three different dimensions (such as in an orthogonal system, in x, y, and z coordinate planes), sensing joint stability as a function of supporting muscle tension, sensing joint acceleration in a joint-segment (a part of a joint and thus having a joint-segment sensor), a joint-region (a portion of a joint or even a joint system such as the upper or lower back, etc. and thus having a joint-region sensor), sensing joint acceleration in a sacroiliac joint, sensing joint acceleration in a ball and socket joint, sensing joint acceleration in a hinge joint, sensing joint acceleration in a condyloid joint, sensing joint acceleration in a pivot joint, sensing joint acceleration in a gliding joint, sensing joint acceleration in a saddle joint, or the like.

In another embodiment, it may be advantageous to further include the step of transmitting stability sensor data when measuring and determining joint stability. In some embodiments, joint stability data may be transmitted to an external application or the like. This external application may be a cellular phone application, computer application, a cloud computing system, or the like.

In another embodiment, it may be beneficial to further include the step of containing a joint stability measurement system with a wearable article. This may be beneficial to allow the joint stability measurement system to be located close to a user's body and joints to aid in the ability to collect viable and accurate movement data. In some embodiments, the containing in a wearable article may include but is not limited to containing a joint stability measurement system or a joint movement sensor with a compression pant, a compression sleeve, a user-adjustable belt, a compression belt, a compression shirt, a sock, or the like.

In some embodiments, the step of calculating joint stability or range—perhaps as a function of joint acceleration and/or joint angular velocity may be accomplished by calculating preferred joint stability or range as a function of joint acceleration and/or joint angular velocity on an external application utilizing at least one joint movement sensor data. In some embodiments, a further step of transmitting at least some joint movement sensor data to a cellular phone application, transmitting at least some joint stability sensor data to a cloud computing system, or the like.

In some embodiments, it may be beneficial to further include the step of actuating a feedback system in response to a computed sustainable joint position or range indication as a function of joint acceleration and/or joint angular velocity. In some embodiments, actuating the feedback system may include actuating a haptic feedback system, actuating a vibrating actuator, actuating an actuator that warms an area of application, actuating a cell phone or the like. In another embodiment, the actuating a feedback system may be accomplished by utilizing a visual indicator such as a light emitting diode, by displaying a notification on an external application, or the like.

In some embodiments, the step of calculating joint stability as a function of joint acceleration and/or joint angular velocity may include calculating a real-time sustainable position joint range, calculating the time between or of unstable and stable joint position, or the like. This may also include times in or out of ranges such as a duration of an in-preferred joint range, a duration of an out-of-preferred joint range, a duration of an unstable joint situation (the time in conditions deemed unstable), and/or a duration of a stable joint situation (the time in conditions deemed stable), and any ratios or such from such information.

In some embodiments, a joint range feedback system, may include but is not limited to a mobile power system, a joint movement sensor (such as may sense any movement-involved aspect—position, velocity, acceleration, force, etc.), a microcontroller configured and arranged to compute joint range as a function of joint acceleration and/or joint angular velocity perhaps in real-time, a range information-based indicator, a joint stability processor, and a joint stability indicator.

A mobile power system may be any source of mobile power and such may include a battery, a capacitor, a piezo-electric power generator, or the like. A mobile power system may be necessary to provide adequate range of motion for a user during certain athletic movements such as running, walking, and/or jumping. A piezo-electric power generator may produce power upon oscillation of piezo-electric components, which may be beneficial to power a joint range measurement system in an active state.

In some embodiments, a joint movement sensor may include but is not limited to a position or location sensor an accelerometer, inertial measurement unit, or the like. In some embodiments, the accelerometer type may be of the piezoresistive, capacitive, or the like accelerometer. In some embodiments, an inertial measurement unit may be of the gyroscopic, magnetic, or the like type of inertial measurement unit. An inertial measurement unit may also include but is not limited to including accelerometers. In some embodiments, an inertial measurement unit may be that of an inertial sensor, inertial measurement unit or system, a gyroscope, an accelerometer, a combination of the aforementioned elements, or the like. An inertial sensor may be beneficial in easily sensing joint rotation and acceleration.

In some embodiments, a microcontroller configured and arranged to compute joint range as a function of joint acceleration and/or joint angular velocity perhaps in real-time may be beneficial. Computing joint range as a function of joint acceleration and joint angular velocity in real-time, or close to real-time (such including in milliseconds, seconds, or coordinated to be accomplished during the specific activity, operation, or movement action involved) may be beneficial in providing optimal joint angle, position, or the like. Real-time, or close to real-time measurement and calculation may be important to measure a user's percentage of time in an unstable movement versus a sustainable movement (or a stable movement).

In some embodiments, the joint movement sensor may sense any type of movement or even just acceleration. This may include an accelerometer, inertial measurement unit, or the like may be configured to measure lateral joint acceleration, lateral joint movement, core muscle stability, joint stability as a function of supporting muscle tension, or the like. In some embodiments, it may be beneficial to take joint acceleration measurements at different types of joints or joint segments. In some embodiments, these joints may be but are not limited to a sacroiliac joint, a ball and socket joint a hinge joint, a condyloid joint, a pivot joint, a gliding joint, a saddle joint, or the like. As an example, measuring joint acceleration over the sacroiliac joint may provide the required data to predict sustainable movement for a user's back health.

In some embodiments, it may be beneficial to include a microcontroller. A microcontroller may be used as a joint stability or movement range processor or to control the transmission of actual data, determined data, or even acceleration data or the like. In some embodiments, the microcontroller may be a computer-controlled chip such as an Arduino, Raspberry Pi, PIC, ASIC, microprocessor, or another microcomputer. In others the microcontroller may be a series of logic circuits, or the like. In some embodiments, it may be beneficial to further include a transmitter that may be used to transmit joint acceleration data to an external application or internal microcontroller. In some embodiments, the external application may be a cellular phone application, comprises a computer application, a cloud computing system, or the like. Data may be processed by an external application or by a microcontroller that in some embodiments, may run a sustainable position joint movement range processor or algorithm. In some embodiments, a sustainable position joint movement range processor may be used to alert a user through haptic feedback of unstable joint movement.

In some embodiments, in order to gather joint acceleration or other data it may be beneficial to contain a joint stability or range measurement system in a wearable containment system. In some embodiments, a user may don a wearable containment system so the joint movement sensor is in close proximity to the joint being measured. In some embodiments, a wearable containment system may be in the form of a compression pant, such as athletic tights, leggings: a compression sleeve, such as an arm sleeve, leg sleeve, calf sleeve, ankle sleeve, sock, or the like: a user-adjustable belt, a compression belt, or the like: or a compression shirt, such as a long sleeve, short sleeve, or tank top: a compression short: a compression underwear, in the form of briefs, trunks, boxer briefs, bikini, thong, or the like: a compression socks: a compression gloves, and the like.

In some embodiments, of a joint movement measurement system, it may be beneficial to further include a user feedback signal. In some embodiments, a user feedback signal may be a haptic feedback signal. Haptic feedback may also be known as kinesthetic communication or three-dimensional touch, and may refer to any technology that can create an experience of touch by applying forces, vibrations, or motions to the user. The haptic feedback may also be the use of advanced vibration patterns and waveforms to convey information to or to alert a user. In some embodiments, a haptic feedback signal may be an actuator configured and arranged to vibrate. In some embodiments, an actuator may be a small DC motor, a piezo-electric actuator, or the like. In some embodiments, an actuator may be configured and arranged to warm the skin of a user. An actuator configured and arranged to warm the skin may be a resistor or resistive actuator that may use electric energy to warm an area of the user's skin in contact with the joint stability measurement system. In other embodiments, a user feedback signal may be a visual indication. In some embodiments, a visual indication may be at least one LED (light emitting diode). In some embodiments, a singular LED may be used and in some embodiments, an array of LEDs may be used. In some embodiments, a user feedback signal may be an external application notification. This external application notification may be broadcast to a phone application, computer application, or the like.

In some embodiments, it may be beneficial to further include a sustainable position joint movement range processor. In some embodiments, a sustainable position joint range processor may be a real-time sustainable position joint range algorithm, or a sustainable position joint range algorithm that calculates the time between unstable and stable joint position, or the like.

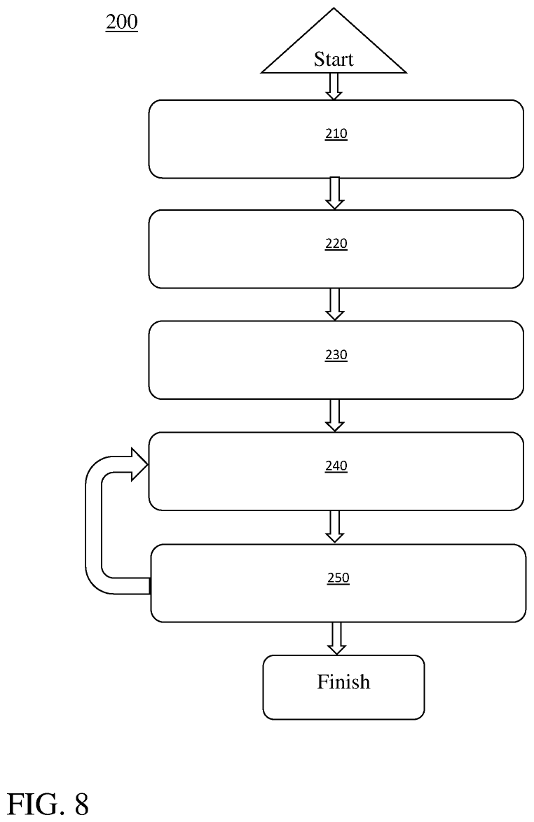

FIG. 8 is an exemplary embodiment, of a method of providing joint range feedback. In some embodiments, it may be beneficial to include a method of providing joint range feedback (200). In some embodiments, a method of providing joint range feedback may be accomplished by portably powering a joint range measurement system (210), providing at least one joint movement sensor (220) (or a sensing system with associated components, and multiple sensors as may exist such as in a sensing system), sensing joint movement with at least one joint movement sensor (230), computing sustainable joint range as a function of joint acceleration and/or joint angular velocity (240), actuating a feedback system in response to the computed sustainable joint range as a function of joint acceleration and/or joint angular velocity (250), and the like.

In some embodiments, portably powering a joint range measurement system may be accomplished by portably powering with a battery, a capacitor, a piezo electric power generator, or the like. A piezo electric power generator may be a series of piezo electric elements that upon introduction of a vibration, movement, or the like generates electricity.

In some embodiments, the step of providing at least one joint stability sensor may be accomplished by providing an accelerometer, either a piezoresistive accelerometer, a capacitive accelerometer, or the like. In other embodiments, providing at least one joint stability sensor may be accomplished by providing an inertial measurement unit. In some embodiments, providing an inertial measurement unit may be accomplished by providing a gyroscopic inertial measurement unit, a magnetic inertial measurement unit, an accelerometer inertial measurement unit, or the like.

In some embodiments, sensing joint stability with said at least one joint stability sensor may be accomplished sensing lateral joint acceleration, sensing lateral joint movement, sensing core muscle stability, sensing joint stability and joint angular velocity in three different dimensions, or in an x, y, and z coordinate planes, sensing joint stability as a function of supporting muscle tension, sensing joint stability in a joint-segment, sensing joint stability in a sacroiliac joint, sensing joint stability in a ball and socket joint, sensing joint stability in a hinge joint, sensing joint stability in a condyloid joint, sensing joint stability in a pivot joint, sensing joint stability in a gliding joint, sensing joint stability in a saddle joint, or the like.

In another embodiment, it may be advantageous to further include the step of transmitting stability sensor data when measuring joint stability. In some embodiments, joint stability data may be transmitted to an external application or the like. This external application may be a cellular phone application, computer application, a cloud computing system, or the like.

In another embodiment, it may be beneficial to further include the step of containing the joint stability measurement system with a wearable article. This may be beneficial to allow the joint stability measurement system to be located close to a user's body and joints to aid in the ability to collect viable and accurate movement data. In some embodiments, the containing in a wearable article may include but is not limited to containing a joint stability measurement system with a compression pant, a compression sleeve, a user-adjustable belt, a compression belt, a compression shirt, a sock, or the like.

In some embodiments, the step of calculating sustainable joint range as a function of joint acceleration and/or joint angular velocity may be accomplished by calculating sustainable joint position as a function of joint acceleration and/or joint angular velocity on an external application utilizing at least some joint stability or movement sensor data. In some embodiments, a further step of transmitting at least some joint movement sensor data to a cellular phone application, transmitting at least one joint stability sensor data to a cloud computing system, or the like.

In some embodiments, it may be beneficial to further include the step of actuating a feedback system in response to a computed sustainable joint range as a function of joint acceleration and/or joint angular velocity. In some embodiments, actuating the feedback system may include actuating a haptic feedback system, actuating a vibrating actuator, actuating an actuator that warms an area of application, or the like. In another embodiment, the actuating a feedback system may be accomplish by utilizing a visual indicator such as a light emitting diode, by displaying a notification on an external application, or the like.

In some embodiments, it may be beneficial to further include the step of calculating joint range as a function of joint acceleration and/or joint angular velocity. In some embodiments, the step of calculating joint stability as a function of joint acceleration and joint angular velocity or the step of calculating a sustainable joint range may include calculating a real-time sustainable position joint range, calculating the time between unstable and stable joint position, or the like.

In some embodiments, an inter-joint measurement system may include a mobile power system, an inter-joint measurement sensor or sensors, a transmitter configured and arranged to transmit inter-joint measurement sensor data, a microcontroller, wherein the microcontroller receives inter-joint measurement sensor data, and the like.

A mobile power system may include a battery, a capacitor, a piezo-electric power generator, or the like. A mobile power system may be necessary to provide adequate range of motion for a user during certain athletic movements such as running, walking, and/or jumping. A piezo-electric power generator may produce power upon oscillation of piezo-electric components, which may be beneficial to power an inter-joint range measurement system in an active state.

In some embodiments, an inter-joint movement sensor may include but is not limited to one or more accelerometers, inertial measurement units, or the like. In some embodiments, the accelerometer type may be of the piezoresistive, capacitive, or the like accelerometer. In some embodiments, an inertial measurement unit may be of the gyroscopic, magnetic, or the like type of inertial measurement unit. An inertial measurement unit may also include but is not limited to including accelerometers. In some embodiments, an inertial measurement unit may be that of an inertial sensor, inertial measurement unit or system, a gyroscope, an accelerometer, a combination of the aforementioned elements, or the like. An inertial sensor may be beneficial in easily sensing inter-joint rotation and acceleration.

In some embodiments, a microcontroller configured and arranged to compute inter-joint range as a function of inter-joint acceleration and inter-joint angular velocity in real-time may be beneficial. Computing inter-joint range as a function of inter-joint acceleration and inter-joint angular velocity in real-time, or close to real-time (such including in milliseconds, seconds, or coordinated to be accomplished during the specific activity, operation, or movement action involved) may be beneficial in providing optimal inter-joint angle, position, or the like determinations. Real-time, or close to real-time measurement and calculation may be important to measure a user's percentage of time in an unstable movement versus a stable movement.

In some embodiments, the inter-joint range or stability sensor that is an accelerometer, inertial measurement unit, or the like may be configured to measure lateral inter-joint acceleration, lateral inter-joint movement, core muscle stability, inter-joint stability as a function of supporting muscle tension, or the like. In some embodiments, it may be beneficial to take inter-joint acceleration or movement measurements at different types of inter-joints or inter-joint segments. In some embodiments, these joints may be but are not limited to a sacroiliac joint, a ball and socket joint a hinge joint, a condyloid joint, a pivot joint, a gliding joint, a saddle joint, or the like. As an example, measuring joint acceleration over the sacroiliac joint may provide the required data to predict sustainable movement for a user's back health.

In some embodiments, it may be beneficial to include a microcontroller. A microcontroller may be used to run an inter-joint stability processor or to control the transmission of movement or acceleration data. In some embodiments, the microcontroller may be a computer-controlled chip such as an Arduino, RaspberryPi, PIC, ASIC, a processor, logic, or another microcomputer. In others the microcontroller may be a series of logic circuits, or the like. In some embodiments, it may be beneficial to further include a transmitter that may be used to transmit inter-joint acceleration data to an external application or internal microcontroller. In some embodiments, the external application may be a cellular phone application, comprises a computer application, a cloud computing system, or the like. Data may be processed by an external application or by a microcontroller that in some embodiments, may run a sustainable position inter-joint range processor. In some embodiments, a sustainable position inter-joint range processor may be used to alert a user through haptic feedback of unstable inter-joint movement determinations.

In some embodiments, in order to gather inter-joint acceleration data, it may be beneficial to contain an inter-joint stability measurement system in a wearable containment system. In some embodiments, a user may don a wearable containment system so the inter-joint stability sensor or sensors is or are in close proximity to the inter-joint or joints being measured. In some embodiments, a wearable containment system may be in the form of a compression pant, such as athletic tights, leggings: a compression sleeve, such as an arm sleeve, leg sleeve, calf sleeve, ankle sleeve, sock, or the like: a user-adjustable belt, a compression belt, or the like: or a compression shirt, such as a long sleeve, short sleeve, or tank top: a compression short: a compression underwear, in the form of briefs, trunks, boxer briefs, bikini, thong, or the like: a compression socks: a compression gloves, and the like.

In some embodiments, of an inter-joint movement, range, or stability measurement system it may be beneficial to further include a user feedback signal. In some embodiments, a user feedback signal may be a haptic feedback signal. Haptic feedback may also be known as kinesthetic communication or three-dimensional touch, and may refer to any technology that can create an experience of touch by applying forces, vibrations, or motions to the user. The haptic feedback may also be the use of advanced vibration patterns and waveforms to convey information to or to alert a user. In some embodiments, a haptic feedback signal may be an actuator configured and arranged to vibrate. In some embodiments, an actuator may be a small DC motor, a piezo-electric actuator, or the like. In some embodiments, an actuator may be configured and arranged to warm the skin of a user. An actuator configured and arranged to warm the skin may be a resistor or resistive actuator that may use electric energy to warm an area of the user's skin in contact with the inter-joint stability measurement system. In other embodiments, a user feedback signal may be a visual indication. In some embodiments, a visual indication may be at least one LED (light-emitting diode). In some embodiments, a singular LED may be used, and in some embodiments, an array of LEDs may be used. In some embodiments, a user feedback signal may be an external application notification. This external application notification may be broadcast to a phone application, computer application, or the like.

In some embodiments, it may be beneficial to further include a sustainable position inter-joint movement range processor. In some embodiments, a sustainable position inter-joint movement range processor may be a real-time sustainable position inter-joint movement range processor, or a sustainable position inter-joint movement range processor that calculates the time between or of unstable and stable inter-joint position, or the like.

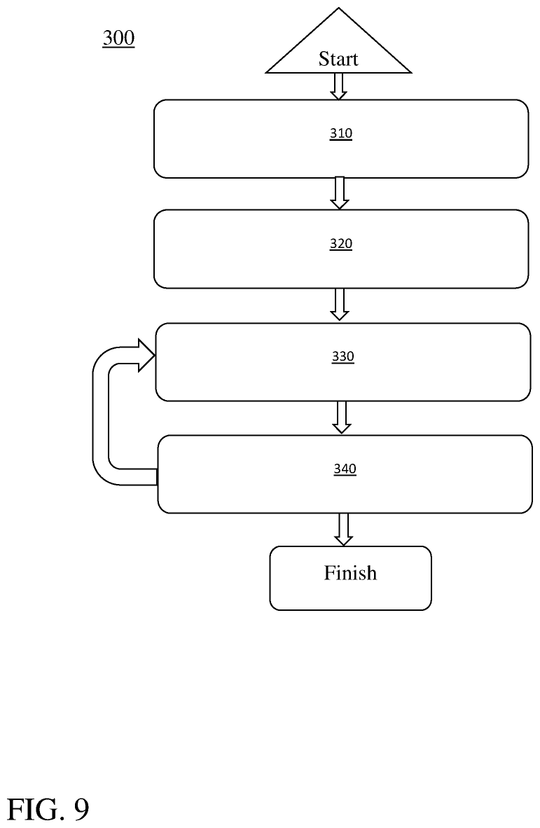

FIG. 9 is an exemplary embodiment, of a method of measuring inter-joint stability. In some embodiments, it may be beneficial to include a method of measuring inter-joint stability (300). In some embodiments, a method of measuring inter-joint stability may be accomplished by portably powering a joint movement measurement system (310), providing at least one inter-joint movement sensor (320), sensing inter-joint movement acceleration with at least one joint movement sensor (330), calculating inter-joint stability as a function of joint acceleration and/or joint angular velocity (340), and the like.

In some embodiments, portably powering an inter-joint acceleration measurement system may be accomplished by portably powering with a battery, a capacitor, a piezo electric power generator, or the like. A piezo electric power generator may be a series of piezo electric elements that, upon introduction of vibration, movement, or the like, generates electricity.

In some embodiments, the step of providing at least one inter-joint stability sensor may be accomplished by providing an accelerometer, either a piezoresistive accelerometer, a capacitive accelerometer, or the like. In other embodiments, providing at least one inter-joint stability sensor may be accomplished by providing an inertial measurement unit. In some embodiments, providing an inertial measurement unit may be accomplished by providing a gyroscopic inertial measurement unit, a magnetic inertial measurement unit, an accelerometer inertial measurement unit, or the like.

In some embodiments, sensing inter-joint movement or acceleration with said at least one inter-joint stability sensor may be accomplished sensing lateral inter-joint acceleration, sensing lateral inter-joint movement, sensing core muscle stability, sensing inter-joint acceleration and inter-joint angular velocity in three different dimensions, or in an x, y, and z coordinate planes, sensing inter-joint stability as a function of supporting muscle activation or tension, sensing inter-joint acceleration in a joint-segment, sensing joint acceleration in a sacroiliac joint, sensing joint acceleration in a ball and socket joint, sensing joint acceleration in a hinge joint, sensing joint acceleration in a condyloid joint, sensing joint acceleration in a pivot joint, sensing joint acceleration in a gliding joint, sensing joint acceleration in a saddle joint, or the like.

In another embodiment, it may be advantageous to further include the step of transmitting stability sensor data when measuring inter-joint stability. In some embodiments, inter-joint stability data may be transmitted to an external application or the like. This external application may be a cellular phone application, computer application, a cloud computing system, or the like.

In another embodiment, it may be beneficial to further include the step of containing the inter-joint movement measurement system with a wearable article. This may be beneficial to allow the inter-joint movement measurement system to be located close to a user's body and inter-joints to aid in the ability to collect viable and accurate movement data. In some embodiments, the containing in a wearable article may include but is not limited to containing an inter-joint stability measurement system with a compression pant, a compression sleeve, a user-adjustable belt, a compression belt, a compression shirt, a sock, or the like.

In some embodiments, the step of calculating inter-joint stability as a function of inter-joint movement or acceleration and/or inter-joint angular velocity may be accomplished by calculating inter-joint stability as a function of inter-joint acceleration and/or inter-joint angular velocity on an external application utilizing at least one inter-joint stability sensor data. In some embodiments, a further step of transmitting at least one inter-joint stability sensor data to a cellular phone application, transmitting at least one inter-joint stability sensor data to a cloud computing system, or the like.

In some embodiments, it may be beneficial to further include the step of actuating a feedback system in response to a computed sustainable inter-joint position as a function of inter-joint acceleration and inter-joint angular velocity. In some embodiments, actuating the feedback system may include actuating a haptic feedback system, actuating a vibrating actuator, actuating an actuator that warms an area of application, or the like. In another embodiment, the actuating a feedback system may be accomplish by utilizing a visual indicator such as a light emitting diode, by displaying a notification on an external application, or the like.

In some embodiments, it may be beneficial to further include the step of calculating inter-joint stability as a function of inter-joint acceleration and/or inter-joint angular velocity. In some embodiments, the step of calculating inter-joint stability as a function of inter-joint acceleration and inter-joint angular velocity may include calculating a real-time sustainable position inter-joint range, calculating the time between unstable and stable inter-joint position, or the like.

While the present invention has been described in connection with some preferred embodiments, it is not intended to limit the scope of the invention to the particular form set forth, but on the contrary, it is intended to cover such alternatives, modifications, and equivalents, as may be included within the spirit and scope of the invention as defined by the statements of inventions. Examples of alternative claims may include:
1. A joint movement wellbeing system comprising:
   a mobile power system;
   a joint movement sensor;
   a microcontroller configured and arranged to determine joint range information as a function of joint acceleration, joint angular velocity, or both; and
   a preferred joint range information-based indicator.
2. A joint movement wellbeing system comprising:
   a mobile power system;
   a joint movement sensor;
   a joint stability processor; and
   a joint stability indicator.
3. A joint movement wellbeing system comprising:
   a mobile power system;
   a joint movement sensor;
   a microcontroller configured and arranged to determine joint range information as a function of joint acceleration, joint angular velocity, or both;
   a joint stability processor;
   a joint stability indicator; and
   a preferred joint range information-based indicator.
4. A joint movement wellbeing system as described in clause 1, 3, or any other clause wherein said microcontroller comprises a real-time joint range processor.
5. A joint movement wellbeing system as described in clause 2, or any other clause wherein said processor comprises a real-time joint stability processor.
6. A joint movement wellbeing system as described in clause 1, 3, or any other clause wherein said joint stability processor comprises a joint stability processor configured and arranged to assess joint stability using aspects selected from:
   joint optimal movement aspects,
   user baseline aspects, and
   consideration of at least one contemporaneously sensed joint movement,
   muscle activation,
   joint muscle activation, and
   core muscle activation.
7. A joint movement wellbeing system as described in clause 3, or any other clause wherein said microcontroller comprises a microcontroller configured and arranged to assess joint range using aspects selected from:
   joint acceleration,
   joint angular velocity,
   joint stability,
   muscle activation,
   joint muscle activation, and
   core muscle activation.
8. A joint movement wellbeing system as described in clause 1, 2, 3, or any other clause wherein said mobile power system comprises a battery.
9. A joint movement wellbeing system as described in clause 1, 2, 3, or any other clause wherein said mobile power system comprises a capacitor.
10. A joint movement wellbeing system as described in clause 1, 2, 3, or any other clause wherein said mobile power system comprises a piezo-electric power generator.
11. A joint movement wellbeing system as described in clause 1, 2, 3, or any other clause wherein said joint movement sensor comprises an accelerometer.
12. A joint movement wellbeing system as described in clause 1, 2, 3, or any other clause wherein said joint movement sensor comprises a sensor configured and arranged to measure joint angular velocity, joint acceleration, or both.
13. A joint movement wellbeing system as described in clause 1, 2, 3, or any other clause wherein said joint movement sensor comprises an accelerometer configured and arranged to measure non-optimal joint movement.
14. A joint movement wellbeing system as described in clause 1, 2, 3, or any other clause wherein said joint movement sensor comprises a sensor system configured and arranged to measure joint movement in three different dimensions.
15. A joint movement wellbeing system as described in clause 1, 2, 3, or any other clause wherein said joint movement sensor comprises a sensor system configured and arranged to measure joint angular velocity in three different dimensions.
16. A joint movement wellbeing system as described in clause 11 or any other clause wherein said accelerometer comprises an accelerometer configured and arranged to measure non-optimal joint movement.
17. A joint movement wellbeing system as described in clause 11 wherein said accelerometer comprises an accelerometer configured and arranged to measure joint movement in three different dimensions.
18. A joint movement wellbeing system as described in clause 11 or any other clause wherein said accelerometer comprises an accelerometer configured and arranged to measure core muscle activation.
19. A joint movement wellbeing system as described in clause 11 or any other clause wherein said accelerometer comprises an accelerometer configured and arranged to measure joint stability as a function of supporting muscle activation.
20. A joint movement wellbeing system as described in clause 11 or any other clause wherein said joint comprises a joint-segment.
21. A joint movement wellbeing system as described in clause 11 or any other clause said joint comprises a sacroiliac joint.
22. A joint movement wellbeing system as described in clause 11 or any other clause wherein said joint comprises a ball and socket joint.
23. A joint movement wellbeing system as described in clause 1, 2, 3 or any other clause wherein said joint comprises a hinge joint.
24. A joint movement wellbeing system as described in clause 1, 2, 3 or any other clause wherein said joint comprises a condyloid joint.
25. A joint movement wellbeing t system as described in clause 1, 2, 3 or any other clause wherein said joint comprises a pivot joint.
26. A joint movement wellbeing system as described in clause 1, 2, 3 or any other clause wherein said joint comprises a gliding joint.
27. A joint movement wellbeing system as described in clause 1, 2, 3 or any other clause wherein said joint comprises a saddle joint.
28. A joint movement wellbeing system as described in clause 1, 2, 3 or any other clause wherein said accelerometer comprises a piezo-resistive accelerometer.
29. A joint movement wellbeing system as described in clause 1, 2, 3 or any other clause wherein said accelerometer comprises a capacitive accelerometer.
30. A joint movement wellbeing system as described in clause 1, 2, 3 or any other clause wherein said joint movement sensor comprises an inertial measurement unit.
31. A joint movement wellbeing system as described in clause 30 or any other clause wherein said inertial measurement unit comprises a gyroscopic inertial measurement unit.
32. A joint movement wellbeing system as described in clause 30 or any other clause wherein said inertial measurement unit comprises a magnetic inertial measurement unit.
33. A joint movement wellbeing system as described in clause 30 or any other clause wherein said inertial measurement unit comprises an inertial measurement unit configured and arranged to measure non-optimal joint acceleration.
34. A joint movement wellbeing system as described in clause 30 or any other clause wherein said inertial measurement unit comprises an inertial measurement unit configured and arranged to measure non-optimal joint movement.
35. A joint movement wellbeing system as described in clause 30 or any other clause wherein said inertial measurement unit comprises an inertial measurement unit configured and arranged to measure joint acceleration and/or joint angular velocity in three different dimensions.
36. A joint movement wellbeing system as described in clause 30 or any other clause wherein said inertial measurement unit comprises an inertial measurement unit configured and arranged to measure core muscle stability.
37. A joint movement wellbeing system as described in clause 30 or any other clause wherein said inertial measurement unit comprises inertial measurement unit configured and arranged to measure joint stability as a function of supporting muscle activation.
38. A joint movement wellbeing system as described in clause 1, 2, 3, or any other clause wherein said joint movement sensor comprises a joint-segment sensor.
39. A joint movement wellbeing system as described in clause 1, 2, 3, or any other clause wherein said joint movement sensor comprises a joint-region sensor.
40. A joint movement wellbeing system as described in clause 1, 2, 3, or any other clause wherein said joint comprises sacroiliac joint.
41. A joint movement wellbeing system as described in clause 1, 2, 3, or any other clause wherein said joint comprises a ball and socket joint.
42. A joint movement wellbeing system as described in clause 1, 2, 3, or any other clause wherein said joint comprises a hinge joint.
43. A joint stability measurement system as described in clause 1, 2, 3, or any other clause wherein said joint comprises a condyloid joint.
44. A joint movement wellbeing system as described in clause 1, 2, 3, or any other clause wherein said joint comprises a pivot joint.
45. A joint movement wellbeing system as described in clause 1, 2, 3, or any other clause wherein said joint comprises a gliding joint.
46. A joint movement wellbeing system as described in clause 1, 2, 3, or any other clause wherein said joint comprises a saddle joint.
47. A joint movement wellbeing system as described in clause 1, 2, 3, or any other clause and further comprising a transmitter.
48. A joint movement wellbeing system as described in clause 47 or any other clause wherein said transmitter comprises a transmitter configured and arranged to transmit data to an external application.
49. A joint movement wellbeing system as described in clause 48 or any other clause wherein said external application comprises a cellular phone application.
50. A joint movement wellbeing system as described in clause 48 or any other clause wherein said external application comprises a computer application.
51. A joint movement wellbeing system as described in clause 48 or any other clause wherein said external application comprises a cloud computing system.
52. A joint movement wellbeing system as described in clause 1, 2, 3, or any other clause further comprising a wearable containment system configured and arranged to contain said joint range measurement system.
53. A joint movement wellbeing system as described in clause 52 or any other clause wherein said wearable containment system comprises a compression pant.
54. A joint movement wellbeing system as described in clause 52 or any other clause wherein said wearable containment system comprises a compression sleeve.
55. A joint movement wellbeing system as described in clause 52 or any other clause wherein said wearable containment system comprises a user-adjustable belt.
56. A joint movement wellbeing system as described in clause 52 or any other clause wherein said wearable containment system comprises a compression belt.
57. A joint movement wellbeing system as described in clause 52 or any other clause wherein said wearable containment system comprises a compression shirt.
58. A joint movement wellbeing system as described in clause 52 or any other clause wherein said wearable containment system comprises a compression short.
59. A joint movement wellbeing system as described in clause 52 or any other clause wherein said wearable containment system comprises a compression underwear.
60. A joint stability measurement system as described in clause 52 or any other clause wherein said wearable containment system comprises a compression sock.
61. A joint movement wellbeing system as described in clause 52 or any other clause wherein said wearable containment system comprises a compression glove.
62. A joint movement wellbeing system as described in clause 1, 3, or any other clause wherein said microcontroller is configured and arranged to utilize a duration of an in-preferred joint range.
63. A joint movement wellbeing system as described in clause 1, 3, or any other clause wherein said microcontroller is configured and arranged to utilize a duration of an out-of-preferred joint range.
64. A joint movement wellbeing system as described in clause 2, 3, or any other clause wherein said joint stability processor is configured and arranged to utilize a duration of an unstable joint determination
65. A joint movement wellbeing system as described in clause 1, 2, 3, or any other clause wherein said joint stability processor is configured and arranged to utilize a duration of a stable joint determination.
66. A joint movement wellbeing system as described in clause 1, 2, 3, or any other clause further comprising a user feedback signal.
67. A joint movement wellbeing system as described in clause 66 or any other clause wherein said user feedback signal comprises a haptic feedback signal.
68. A joint movement wellbeing system as described in clause 67 or any other clause wherein said haptic feedback signal comprises an actuator configured and arranged to vibrate.
69. A joint movement wellbeing system as described in clause 67 or any other clause wherein said haptic feedback signal comprises an actuator configured and arranged to warm the skin of a user.
70. A joint movement wellbeing system as described in clause 66 or any other clause wherein said user feedback signal comprises a visual indication
71. A joint movement wellbeing system as described in clause 70 or any other clause wherein said visual indication comprises an at least one LED.
72. A joint movement wellbeing system as described in clause 66 or any other clause wherein said user feedback signal comprises an external application notification.
73. A joint range feedback system comprising:
   a mobile power system;
   a joint movement sensor;
   a joint movement range processor: and
   a user feedback signal.
74. A joint range feedback system as described in clause 73 or any other clause wherein said mobile power system comprises a battery.
75. A joint range feedback system as described in clause 73 or any other clause wherein said mobile power system comprises a capacitor.
76. A joint range feedback system as described in clause 73 or any other clause wherein said mobile power system comprises a piezoelectric power generator.
77. A joint range feedback system as described in clause 73 or any other clause wherein said joint range sensor comprises an accelerometer.
78. A joint range feedback system as described in clause 77 or any other clause wherein said accelerometer comprises an accelerometer configured and arranged to measure non-optimal joint acceleration.
79. A joint range feedback system as described in clause 77 or any other clause wherein said accelerometer comprises an accelerometer configured and arranged to measure non-optimal joint movement.
80. A joint range feedback system as described in clause 77 or any other clause wherein said accelerometer comprises an accelerometer configured and arranged to measure core muscle activation.
81. A joint range feedback system as described in clause 77 or any other clause wherein said accelerometer comprises an accelerometer configured and arranged to measure joint stability as a function of supporting muscle activation.
82. A joint range feedback system as described in clause 77 or any other clause wherein said joint comprises a joint-segment.
83. A joint range feedback system as described in clause 77 or any other clause wherein said joint comprises a sacroiliac joint.
84. A joint range feedback system as described in clause 77 or any other clause wherein said joint comprises a ball and socket joint.
85. A joint range feedback system as described in clause 77 or any other clause wherein said joint comprises a hinge joint.
86. A joint range feedback system as described in clause 77 or any other clause wherein said joint comprises a condyloid joint
87. A joint range feedback system as described in clause 77 or any other clause wherein said joint comprises a pivot joint.
88. A joint range feedback system as described in clause 77 or any other clause wherein said joint comprises a gliding joint.
89. A joint range feedback system as described in clause 77 or any other clause wherein said joint comprises a saddle joint.
90. A joint range feedback system as described in clause 77 or any other clause wherein said accelerometer comprises a piezoresistive accelerometer.
91. A joint range feedback system as described in clause 77 or any other clause wherein said accelerometer comprises a capacitive accelerometer.
92. A joint range feedback system as described in clause 77 or any other clause wherein said joint range sensor comprises an inertial measurement unit.
93. A joint range feedback system as described in clause 92 or any other clause wherein said inertial measurement unit comprises a gyroscopic inertial measurement unit.
94. A joint range feedback system as described in clause 92 or any other clause wherein said inertial measurement unit comprises a magnetic inertial measurement unit.
95. A joint range feedback system as described in clause 92 or any other clause wherein said inertial measurement unit comprises an inertial measurement unit configured and arranged to measure non-optimal joint acceleration.
96. A joint range feedback system as described in clause 92 or any other clause wherein said inertial measurement unit comprises an inertial measurement unit configured and arranged to measure non-optimal joint movement.
97. A joint range feedback system as described in clause 92 or any other clause wherein said inertial measurement unit comprises an inertial measurement unit configured and arranged to measure core muscle stability.
98. A joint range feedback system as described in clause 92 or any other clause wherein said inertial measurement unit comprises an inertial measurement unit configured and arranged to measure joint stability as a function of supporting muscle activation.
99. A joint range feedback system as described in clause 92 or any other clause wherein said joint comprises a joint-segment.
100. A joint range feedback system as described in clause 99 or any other clause wherein said joint comprises a sacroiliac joint.
101. A joint range feedback system as described in clause 92 or any other clause wherein said joint comprises a ball and socket joint.
102. A joint range feedback system as described in clause 92 or any other clause wherein said joint comprises a hinge joint.
103. A joint range feedback system as described in clause 92 or any other clause wherein said joint comprises a condyloid joint.
104. A joint range feedback system as described in clause 92 or any other clause wherein said joint comprises a pivot joint.
105. A joint range feedback system as described in clause 92 or any other clause wherein said joint comprises a gliding joint.
106. A joint range feedback system as described in clause 92 or any other clause wherein said joint comprises a saddle joint.
107. A joint range feedback system as described in clause 73 or any other clause wherein said processor comprises a processor configured and arranged to calculate joint range as function of joint acceleration.
108. A joint range feedback system as described in clause 107 or any other clause further comprising a transmitter.
109. A joint range feedback system as described in clause 108 or any other clause wherein said transmitter comprises a transmitter configured and arranged to send data to an external application.
110. A joint range feedback system as described in clause 109 or any other clause wherein said external application comprises a cellular phone application.
111. A joint range feedback system as described in clause 109 or any other clause wherein said external application comprises a computer application.
112. A joint range feedback system as described in clause 109 or any other clause wherein said external application comprises a cloud computing system.
113. A joint range feedback system as described in clause 73 or any other clause further comprises a wearable containment system configured and arranged to contain said joint range feedback system.
114. A joint range feedback system as described in clause 113 or any other clause wherein said wearable containment system comprises a compression pant.
115. A joint range feedback system as described in clause 113 or any other clause wherein said wearable containment system comprises a compression sleeve.
116. A joint range feedback system as described in clause 113 or any other clause wherein said wearable containment system comprises a user-adjustable belt.
117. A joint range feedback system as described in clause 113 or any other clause wherein said wearable containment system comprises a compression belt.
118. A joint range feedback system as described in clause 113 or any other clause wherein said wearable containment system comprises a compression shirt.
119. A joint range feedback system as described in clause 73 or any other clause wherein said user feedback signal comprises a haptic feedback signal.
120. A joint range feedback system as described in clause 119 or any other clause wherein said haptic feedback signal comprises an actuator configured and arranged to vibrate.
121. A joint range feedback system as described in clause 119 or any other clause wherein said haptic feedback signal comprises an actuator configured and arranged to warm the skin of a user.
122. A joint range feedback system as described in clause 73 or any other clause wherein said user feedback signal comprises a visual indication
123. A joint range feedback system as described in 122 or any other clause wherein said visual indication comprises an at least one light emitting diode.
124. A joint range feedback system as described in clause 73 or any other clause wherein said user feedback signal comprises an external application notification.
125. A joint range feedback system as described in clause 73 or any other clause further comprising a real-time joint movement range processor.
126. A joint range feedback system as described in clause 125 or any other clause wherein said real-time joint movement range processor comprises a real-time sustainable joint movement range processor.
127. A joint range feedback system as described in clause 125 or any other clause wherein said real-time joint movement range processor comprises a processor that calculates the time between unstable and stable joint position.
128. An inter-joint measurement system comprising:
   a mobile power system;
   an inter-joint measurement sensor:
      a transmitter configured and arranged to transmit inter-joint measurement sensor data: and
   a microcontroller, wherein said microcontroller receives inter-joint measurement sensor data.
129. An inter-joint measurement system as described in clause 128 or any other clause wherein said mobile power system comprises a battery.
130. An inter-joint measurement system as described in clause 128 or any other clause wherein said mobile power system comprises a capacitor.
131. An inter-joint measurement system as described in clause 128 or any other clause wherein said mobile power system comprises piezo-electric power generator.
132. An inter-joint measurement system as described in clause 128 or any other clause wherein said inter-joint measurement sensor comprises an accelerometer.
133. An inter-joint measurement system as described in clause 132 or any other clause wherein said accelerometer comprises an accelerometer configured and arranged to measure non-optimal joint acceleration.
134. An inter-joint measurement system as described in clause 132 or any other clause wherein said accelerometer comprises an accelerometer configured and arranged to measure non-optimal joint movement.
135. An inter-joint measurement system as described in clause 132 or any other clause wherein said accelerometer comprises an accelerometer configured and arranged to measure core muscle activation.
136. An inter-joint measurement system as described in clause 132 or any other clause wherein said accelerometer comprises an accelerometer configured and arranged to measure joint stability as a function of supporting muscle activation.
137. An inter-joint measurement system as described in clause 132 or any other clause wherein said joint comprises a joint-segment.
138. An inter-joint measurement system as described in clause 132 or any other clause wherein said joint comprises a sacroiliac joint.
139. An inter-joint measurement system as described in clause 132 or any other clause wherein said joint comprises a ball and socket joint.
140. An inter-joint measurement system as described in clause 132 or any other clause wherein said joint comprises a hinge joint.
141. An inter-joint measurement system as described in clause 132 or any other clause wherein said joint comprises a condyloid joint.
142. An inter-joint measurement system as described in clause 132 or any other clause wherein said joint comprises a pivot joint.
143. An inter-joint measurement system as described in clause 132 or any other clause wherein said joint comprises a gliding joint.
144. An inter-joint measurement system as described in clause 132 or any other clause wherein said joint comprises a saddle joint.
145. An inter-joint measurement system as described in clause 132 or any other clause wherein said accelerometer comprises a piezo-resistive accelerometer.
146. An inter-joint measurement system as described in clause 132 or any other clause wherein said accelerometer comprises a capacitive accelerometer.
147. An inter-joint measurement system as described in clause 128 or any other clause wherein said joint movement sensor comprises an inertial measurement unit
148. An inter-joint measurement system as described in clause 147 or any other clause wherein said inertial measurement unit comprises a gyroscopic inertial measurement unit.
149. An inter-joint measurement system as described in clause 147 or any other clause wherein said inertial measurement unit comprises a magnetic inertial measurement unit.
150. An inter-joint measurement system as described in clause 147 wherein said inertial measurement unit comprises an accelerometer inertial measurement unit.
151. An inter-joint measurement system as described in clause 147 or any other clause wherein said inertial measurement unit comprises an inertial measurement unit configured and arranged to measure non-optimal joint acceleration.
152. An inter-joint measurement system as described in clause 147 or any other clause wherein said inertial measurement unit comprises an inertial measurement unit configured and arranged to measure non-optimal joint movement.
153. An inter-joint measurement system as described in clause 147 or any other clause wherein said inertial measurement unit comprises an inertial measurement unit configured and arranged to measure core muscle stability.
154. An inter-joint measurement system as described in clause 147 or any other clause wherein said inertial measurement unit comprises inertial measurement unit configured and arranged to measure joint stability as a function of supporting muscle activation.
155. An inter-joint measurement system as described in clause 147 or any other clause wherein said joint comprises joint-segment.
156. An inter-joint measurement system as described in clause 155 or any other clause wherein said joint comprises sacroiliac joint.
157. An inter-joint measurement system as described in clause 147 or any other clause wherein said joint comprises a ball and socket joint.
158. An inter-joint measurement system as described in clause 147 or any other clause wherein said joint comprises a hinge joint.
159. An inter-joint measurement system as described in clause 147 or any other clause wherein said joint comprises a condyloid joint.
160. An inter-joint measurement system as described in clause 147 or any other clause wherein said joint comprises a pivot joint.
161. An inter-joint measurement system as described in clause 147 or any other clause wherein said joint comprises a gliding joint.
162. An inter-joint measurement system as described in clause 147 or any other clause wherein said joint comprises a saddle joint.
163. An inter-joint measurement system as described in clause 128 or any other clause further comprising a transmitter.
164. An inter-joint measurement system as described in clause 163 or any other clause wherein said transmitter comprises a transmitter configured and arranged to transmit data to an external application.
165. An inter-joint measurement system as described in clause 164 or any other clause wherein said external application comprises a cellular phone application.
166. An inter-joint measurement system as described in clause 164 or any other clause wherein said external application comprises a computer application.
167. An inter-joint measurement system as described in clause 164 or any other clause wherein said external application comprises a cloud computing system.
168. An inter-joint measurement system as described in clause 128 or any other clause further comprises a wearable containment system configured and arranged to contain said joint range measurement system.
169. An inter-joint measurement system as described in clause 168 or any other clause wherein said wearable containment system comprises a compression pant.
170. An inter-joint measurement system as described in clause 168 or any other clause wherein said wearable containment system comprises a compression sleeve.
171. An inter-joint measurement system as described in clause 168 or any other clause wherein said wearable containment system comprises a user-adjustable belt.
172. An inter-joint measurement system as described in clause 168 or any other clause wherein said wearable containment system comprises a compression belt.
173. An inter-joint measurement system as described in clause 168 or any other clause wherein said wearable containment system comprises a compression shirt.
174. An inter-joint measurement system as described in clause 128 or any other clause further comprising a joint movement range processor.
175. An inter-joint measurement system as described in clause 174 or any other clause wherein said joint movement range processor comprises a real-time joint movement range processor.
176. An inter-joint measurement system as described in clause 174 or any other clause wherein said joint movement range processor comprises a joint movement range processor that calculates the time between unstable and stable joint position.
177. A method of considering joint wellbeing comprising the steps of:
   portably powering a joint movement sensing system;
   sensing joint movement by said at least one joint movement sensor system;
   determining preferred joint range information as a function of joint acceleration, joint angular velocity, or both; and
   indicating information based on said preferred joint range.
178. A method of considering joint wellbeing comprising the steps of:
   portably powering a joint movement sensing system;
   sensing joint movement by said at least one joint movement sensor system;
   determining joint stability; and
   indicating joint stability.
179. A method of considering joint wellbeing as described in clause 177 or any other clause and further comprising the steps of:
   determining joint stability: and
   indicating joint stability.
180. A method of considering joint wellbeing as described in clause 177, 178 or any other clause wherein said step of portably powering a joint acceleration measurement system comprises portably powering with a battery.
181. A method of considering joint wellbeing as described in clause 177, 178 or any other clause wherein said step of portably powering a joint acceleration measurement system comprises portably powering with a capacitor.
182. A method of considering joint wellbeing as described in clause 177, 178 or any other clause wherein said step of portably powering a joint acceleration measurement system comprises portably powering with a piezo electric power generator.
183. A method of considering joint wellbeing as described in clause 177, 178 or any other clause wherein said step of providing at least one joint stability sensor comprises providing an accelerometer.
184. A method of considering joint wellbeing as described in clause 177, 178 or any other clause wherein said step providing an accelerometer comprises providing a piezoresistive accelerometer.
185. A method of considering joint wellbeing as described in clause 177, 178 or any other clause wherein said step providing an accelerometer comprises providing a capacitive accelerometer.
186. A method of considering joint wellbeing as described in clause 177, 178 or any other clause wherein said step of sensing joint stability with said at least one joint stability sensor comprises sensing non-optimal joint acceleration.
187. A method of considering joint wellbeing as described in clause 177, 178 or any other clause wherein said step of sensing joint stability with said at least one joint stability sensor comprises sensing non-optimal joint movement.
188. A method of considering joint wellbeing as described in clause 177, 178 or any other clause wherein said step of sensing joint stability with said at least one joint stability sensor comprises sensing core muscle activation.
189. A method of considering joint wellbeing as described in clause 177, 178 or any other clause wherein said step of sensing joint stability with said at least one joint stability sensor comprises sensing joint stability and/or joint angular velocity in three different dimensions.
190. A method of considering joint wellbeing as described in clause 177, 178 or any other clause wherein said step of sensing joint stability with said at least one joint stability sensor comprises sensing joint stability as a function of supporting muscle activation.
191. A method of considering joint wellbeing as described in clause 177, 178 or any other clause wherein said step of sensing joint stability with said at least one joint stability sensor comprises sensing joint stability in a joint-segment.
192. A method of considering joint wellbeing as described in clause 177, 178 or any other clause wherein said step of sensing joint stability with said at least one joint stability sensor comprises sensing joint stability in a sacroiliac joint.
193. A method of considering joint wellbeing as described in clause 177, 178 or any other clause wherein said step of sensing joint stability with said at least one joint stability sensor comprises sensing joint stability in a ball and socket joint.
194. A method of considering joint wellbeing as described in clause 177, 178 or any other clause wherein said step of sensing joint stability with said at least one joint stability sensor comprises sensing joint stability in a hinge joint.
195. A method of considering joint wellbeing as described in clause 177, 178 or any other clause wherein said step of sensing joint stability with said at least one joint stability sensor comprises sensing joint stability in a condyloid joint.
196. A method of considering joint wellbeing as described in clause 177, 178 or any other clause wherein said step of sensing joint stability with said at least one joint stability sensor comprises sensing joint stability in a pivot joint.
197. A method of considering joint wellbeing as described in clause 177, 178 or any other clause wherein said step of sensing joint stability with said at least one joint stability sensor comprises sensing joint stability in a gliding joint.
198. A method of considering joint wellbeing as described in clause 177, 178 or any other clause wherein said step of sensing joint stability with said at least one joint stability sensor comprises sensing joint stability in a saddle joint.
199. A method of considering joint wellbeing as described in clause 177, 178 or any other clause wherein said step of providing at least one joint stability sensor comprises providing an inertial measurement unit.
200. A method of considering joint wellbeing as described in clause 199 or any other clause wherein said step of providing an inertial measurement unit comprises providing a gyroscopic inertial measurement unit.
201. A method of considering joint wellbeing as described in clause 199 or any other clause wherein said step of providing an inertial measurement unit comprises providing a magnetic inertial measurement unit.
202. A method of considering joint wellbeing as described in clause 177, 178 or any other clause further comprising the step of transmitting said at least one joint stability sensor data.
203. A method of considering joint wellbeing as described in clause 202 or any other clause wherein the step of transmitting said at least one joint stability sensor data comprises transmitting said at least one joint stability sensor data to an external application.
204. A method of considering joint wellbeing as described in clause 203 or any other clause wherein said step of transmitting said at least one joint stability sensor data to an external application comprises transmitting at least one joint stability sensor data to a cellular phone application.
205. A method of considering joint wellbeing as described in clause 203 or any other clause wherein said step of transmitting said at least one joint stability sensor data to an external application comprises transmitting at least one joint stability sensor data to a computer application.
206. A method of considering joint wellbeing as described in clause 203 or any other clause wherein said step of transmitting said at least one joint stability sensor data to an external application comprises transmitting at least one joint stability sensor data to a cloud computing system.
207. A method of considering joint wellbeing as described in clause 177, 178 or any other clause further comprises the step of containing a joint movement measurement system with a wearable containment system.
208. A method of considering joint wellbeing as described in clause 207 or any other clause wherein said step of containing a joint stability measurement system comprises containing said joint stability measurement system with a compression pant.
209. A method of considering joint wellbeing as described in clause 207 or any other clause wherein said step of containing a joint stability measurement system comprises containing said joint stability measurement system with a compression sleeve.
210. A method of considering joint wellbeing as described in clause 207 or any other clause wherein said step of containing a joint stability measurement system comprises containing said joint stability measurement system with a user-adjustable belt.
211. A method of considering joint wellbeing as described in clause 207 or any other clause wherein said step of containing a joint stability measurement system comprises containing said joint stability measurement system with a compression belt.
212. A method of considering joint wellbeing as described in clause 207 or any other clause wherein said step of containing a joint stability measurement system comprises containing said joint stability measurement system with a compression shirt.
213. A method of considering joint wellbeing as described in clause 177, 178 or any other clause wherein said step of calculating joint stability as a function of joint acceleration and/or joint angular velocity comprises calculating joint stability as a function of joint acceleration and/or joint angular velocity on an external application utilizing said at least one joint stability sensor data.
214. A method of considering joint wellbeing as described in clause 213 or any other clause further comprising transmitting at least one joint stability sensor data to a cellular phone application.
215. A method of considering joint wellbeing as described in clause 213 or any other clause further comprising transmitting at least one joint stability sensor data to a computer application.
216. A method of considering joint wellbeing as described in clause 213 or any other clause further comprising transmitting at least one joint stability sensor data to a cloud computing system.
217. A method of considering joint wellbeing as described in clause 177, 178 or any other clause further comprising a step of actuating a feedback system in response to said step of determining preferred joint range information as a function of joint acceleration, joint angular velocity, or both pr said step of determining joint stability.
218. A method of considering joint wellbeing as described in clause 217 or any other clause wherein said step of actuating a feedback system in response to said step of determining preferred joint range information as a function of joint acceleration, joint angular velocity, or both pr said step of determining joint stability comprises actuating a haptic feedback system.
219. A method of considering joint wellbeing as described in clause 218 or any other clause wherein said step of actuating a haptic feedback system comprises actuating a vibrating actuator.
220. A method of considering joint wellbeing as described in clause 218 or any other clause wherein said step of actuating a haptic feedback system comprises actuating an actuator that warms the skin of a user.
221. A method of considering joint wellbeing as described in clause 217 or any other clause wherein said step of actuating a feedback system in response to said step of determining preferred joint range information as a function of joint acceleration, joint angular velocity, or both pr said step of determining joint stability comprises utilizing a visual indicator.
222. A method of considering joint wellbeing as described in clause 221 or any other clause wherein said step of utilizing a visual indicator comprises illuminating an at least one light emitting diode.
223. A method of considering joint wellbeing as described in clause 217 or any other clause wherein said step of utilizing a visual indicator comprises displaying a notification on an external application.
224. A method of considering joint wellbeing as described in clause 177, 178 or any other clause wherein said step of calculating joint stability as a function of joint acceleration and/or joint angular velocity comprises calculating a real-time preferred joint movement range.
225. A method of considering joint wellbeing described in clause 177, 178 or any other clause wherein said step of calculating joint stability as a function of joint acceleration and/or joint angular velocity comprises calculating the time between unstable and stable joint position.
226. A method of providing joint range feedback comprising the steps of:
   portably powering a joint range measurement system;
   providing at least one joint stability sensor;
   sensing joint stability with said at least one joint stability sensor;
   calculating joint movement range information as a function of joint acceleration and/or joint angular velocity;
   actuating a feedback system in response to said step of calculating joint movement range information as a function of joint acceleration and/or joint angular velocity.
227. A method of providing joint range feedback as described in clause 226 or any other clause wherein said step of portably powering a joint stability measurement system comprises portably powering with a battery.
228. A method of providing joint range feedback as described in clause 226 or any other clause wherein said step of portably powering a joint stability measurement system comprises portably powering with a capacitor.
229. A method of providing joint range feedback as described in clause 226 or any other clause wherein said step of portably powering a joint stability measurement system comprises portably powering with a piezo electric power generator.
230. A method of providing joint range feedback as described in clause 226 or any other clause wherein said step of providing at least one joint stability sensor comprises providing an accelerometer.
231. A method of providing joint range feedback as described in clause 226 or any other clause wherein said step providing an accelerometer comprises providing a piezoresistive accelerometer.
232. A method of providing joint range feedback as described in clause 226 or any other clause wherein said step providing an accelerometer comprises providing a capacitive accelerometer.
233. A method of providing joint range feedback as described in clause 226 or any other clause wherein said step of sensing joint stability with said at least one joint stability sensor comprises sensing non-optimal joint acceleration.
234. A method of providing joint range feedback as described in clause 226 or any other clause wherein said step of sensing joint stability with said at least one joint stability sensor comprises sensing non-optimal joint movement.
235. A method of providing joint range feedback as described in clause 226 or any other clause wherein said step of sensing joint stability with said at least one joint stability sensor comprises sensing core muscle activation.
236. A method of providing joint range feedback as described in clause 226 or any other clause wherein said step of sensing joint stability with said at least one joint stability sensor comprises sensing joint stability as a function of supporting muscle activation.
237. A method of providing joint range feedback as described in clause 226 or any other clause wherein said step of sensing joint stability with said at least one joint stability sensor comprises sensing joint stability in a joint-segment.
238. A method of providing joint range feedback as described in clause 226 or any other clause wherein said step of sensing joint stability with said at least one joint stability sensor comprises sensing joint stability in a sacroiliac joint.
239. A method of providing joint range feedback as described in clause 226 or any other clause wherein said step of sensing joint stability with said at least one joint stability sensor comprises sensing joint stability in a ball and socket joint.
240. A method of providing joint range feedback as described in clause 226 or any other clause wherein said step of sensing joint stability with said at least one joint stability sensor comprises sensing joint stability in a hinge joint.
241. A method of providing joint range feedback as described in clause 226 or any other clause wherein said step of sensing joint stability with said at least one joint stability sensor comprises sensing joint stability in a condyloid joint.
242. A method of providing joint range feedback as described in clause 226 or any other clause wherein said step of sensing joint stability with said at least one joint stability sensor comprises sensing joint stability in a pivot joint.
243. A method of providing joint range feedback as described in clause 226 or any other clause wherein said step of sensing joint stability with said at least one joint stability sensor comprises sensing joint stability in a gliding joint.
244. A method of providing joint range feedback as described in clause 226 or any other clause wherein said step of sensing joint stability with said at least one joint stability sensor comprises sensing joint stability in a saddle joint.
245. A method of providing joint range feedback as described in clause 226 or any other clause wherein said step of providing at least one joint stability sensor comprises providing an inertial measurement unit.
246. A method of providing joint range feedback as described in clause 245 or any other clause wherein said step of providing an inertial measurement unit comprises providing a gyroscopic inertial measurement unit.
247. A method of providing joint range feedback as described in clause 245 or any other clause wherein said step of providing an inertial measurement unit comprises providing a magnetic inertial measurement unit.
248. A method of providing joint range feedback as described in clause 226 or any other clause further comprising the step of transmitting said at least one joint stability sensor data.
249. A method of providing joint range feedback as described in clause 248 or any other clause wherein the step of transmitting said at least one joint stability sensor data comprises transmitting said at least one joint stability sensor data to an external application.
250. A method of providing joint range feedback as described in clause 249 or any other clause wherein said step of transmitting said at least one joint stability sensor data to an external application comprises transmitting at least one joint stability sensor data to a cellular phone application.
251. A method of providing joint range feedback as described in clause 249 or any other clause wherein said step of transmitting said at least one joint stability sensor data to an external application comprises transmitting at least one joint stability sensor data to a computer application.
252. A method of providing joint range feedback as described in clause 249 or any other clause wherein said step of transmitting said at least one joint stability sensor data to an external application comprises transmitting at least one joint stability sensor data to a cloud computing system.
253. A method of providing joint range feedback as described in clause 226 or any other clause further comprises the step of containing a joint stability measurement system with a wearable containment system.
254. A method of providing joint range feedback as described in clause 253 or any other clause wherein said step of containing a joint stability measurement system comprises containing said joint stability measurement system with a compression pant.
255. A method of providing joint range feedback as described in clause 253 or any other clause wherein said step of containing a joint stability measurement system comprises containing said joint stability measurement system with a compression sleeve.
256. A method of providing joint range feedback as described in clause 253 or any other clause wherein said step of containing a joint stability measurement system comprises containing said joint stability measurement system with a user-adjustable belt.
257. A method of providing joint range feedback as described in clause 253 or any other clause wherein said step of containing a joint stability measurement system comprises containing said joint stability measurement system with a compression belt.
258. A method of providing joint range feedback as described in clause 253 or any other clause wherein said step of containing a joint stability measurement system comprises containing said joint stability measurement system with a compression shirt.
259. A method of providing joint range feedback as described in clause 226 or any other clause wherein said step of calculating joint range as a function of joint acceleration and/or joint angular velocity comprises calculating joint range as a function of joint acceleration and/or joint angular velocity on an external application utilizing said at least one joint stability sensor data.
260. A method of providing joint range feedback as described in clause 259 or any other clause wherein said step of calculating joint range as a function of joint acceleration and/or joint angular velocity on an external application utilizing said at least one joint stability sensor data comprises transmitting at least one joint stability sensor data to a cellular phone application.
261. A method of providing joint range feedback as described in clause 259 or any other clause wherein said step of calculating joint range as a function of joint acceleration and/or joint angular velocity on an external application utilizing said at least one joint stability sensor data comprises transmitting at least one joint stability sensor data to a computer application.
262. A method of providing joint range feedback as described in clause 259 or any other clause wherein said step of calculating joint range as a function of joint acceleration and/or joint angular velocity on an external application utilizing said at least one joint stability sensor data comprises transmitting at least one joint stability sensor data to a cloud computing system.
263. A method of providing joint range feedback as described in clause 226 or any other clause wherein said step of actuating a feedback system in response to said step of calculating joint movement range information as a function of joint acceleration and/or joint angular velocity comprises actuating a haptic feedback system.
264. A method of providing joint range feedback as described in clause 263 or any other clause wherein said step of actuating a haptic feedback system comprises actuating a vibrating actuator.
265. A method of providing joint range feedback as described in clause 263 or any other clause wherein said step of actuating a haptic feedback system comprises actuating an actuator that warms the skin of a user.
266. A method of providing joint range feedback as described in clause 226 or any other clause wherein said step of actuating a feedback system in response to said step of calculating joint movement range information as a function of joint acceleration and/or joint angular velocity comprises utilizing a visual indicator.
267. A method of providing joint range feedback as described in clause 266 or any other clause wherein said step of utilizing a visual indicator comprises illuminating an at least one light emitting diode.
268. A method of providing joint range feedback as described in clause 226 or any other clause wherein said step of utilizing a visual indicator comprises displaying a notification on an external application.
269. A method of providing joint range feedback as described in clause 226 or any other clause further comprising the step of calculating joint stability as a function of joint acceleration and/or joint angular velocity.
270. A method of providing joint range feedback as described in clause 226 or any other clause wherein said step of calculating joint stability as a function of joint acceleration and/or joint angular velocity comprises calculating a real-time sustainable position joint range.
271. A method of providing joint range feedback as described in clause 269 or any other clause wherein said step of calculating joint stability as a function of joint acceleration and/or joint angular velocity comprises calculating the time between unstable and stable joint position.
272. A method of measuring inter-joint stability comprising the steps of:
   portably powering an inter-joint stability measurement system;
   providing at least one inter-joint stability sensor: and
   measuring inter-joint acceleration and inter-joint angular velocity with said at least one inter-joint stability sensor.
273. A method of measuring inter-joint stability as described in clause 272 or any other clause wherein said step of portably powering an inter-joint stability measurement system comprises portably powering with a battery.
274. A method of measuring inter-joint stability as described in clause 272 or any other clause wherein said step of portably powering an inter-joint stability measurement system comprises portably powering with a capacitor.
275. A method of measuring inter-joint stability as described in clause 272 or any other clause wherein said step of portably powering an inter-joint stability measurement system comprises portably powering with a piezoelectric power generator.
276. A method of measuring inter-joint stability as described in clause 272 or any other clause wherein said step of providing at least one inter-joint stability sensor comprises providing an accelerometer.
277. A method of measuring inter-joint stability as described in clause 272 or any other clause wherein said step providing an accelerometer comprises providing a piezoresistive accelerometer.
278. A method of measuring inter-joint stability as described in clause 272 or any other clause wherein said step providing an accelerometer comprises providing a capacitive accelerometer.
279. A method of measuring inter-joint stability as described in clause 272 or any other clause wherein said step of sensing joint stability with said at least one inter-joint stability sensor comprises sensing non-optimal joint acceleration.
280. A method of measuring inter-joint stability as described in clause 272 or any other clause wherein said step of sensing inter-joint acceleration with said at least one inter-joint stability sensor comprises sensing non-optimal joint movement.
281. A method of measuring inter-joint stability as described in clause 272 or any other clause wherein said step of sensing inter-joint acceleration with said at least one inter-joint stability sensor comprises sensing core muscle activation.
282. A method of measuring inter-joint stability as described in clause 272 or any other clause wherein said step of sensing inter-joint acceleration with said at least one inter-joint stability sensor comprises sensing inter-joint stability as a function of supporting muscle activation.
283. A method of measuring inter-joint stability as described in clause 272 or any other clause wherein said step of sensing inter-joint acceleration with said at least one inter-joint stability sensor comprises sensing inter-joint acceleration in a joint-segment.
284. A method of measuring inter-joint stability as described in clause 272 or any other clause wherein said step of sensing inter-joint acceleration with said at least one inter-joint stability sensor comprises sensing inter-joint acceleration in a sacroiliac joint.
285. A method of measuring inter-joint stability as described in clause 272 or any other clause wherein said step of sensing inter-joint acceleration with said at least one inter-joint stability sensor comprises sensing inter-joint acceleration in a ball and socket joint.
286. A method of measuring inter-joint stability as described in clause 272 or any other clause wherein said step of sensing inter-joint acceleration with said at least one inter-joint stability sensor comprises sensing inter-joint acceleration in a hinge joint.
287. A method of measuring inter-joint stability as described in clause 272 or any other clause wherein said step of sensing inter-joint acceleration with said at least one inter-joint stability sensor comprises sensing inter-joint acceleration in a condyloid joint.
288. A method of measuring inter-joint stability as described in clause 272 or any other clause wherein said step of sensing inter-joint acceleration with said at least one inter-joint stability sensor comprises sensing inter-joint acceleration in a pivot joint.
289. A method of measuring inter-joint stability as described in clause 272 or any other clause wherein said step of sensing inter-joint acceleration with said at least one inter-joint stability sensor comprises sensing inter-joint acceleration in a gliding joint.
290. A method of measuring inter-joint stability as described in clause 272 or any other clause wherein said step of sensing inter-joint acceleration with said at least one inter-joint stability sensor comprises sensing inter-joint acceleration in a saddle joint.
291. A method of measuring inter-joint stability as described in clause 272 or any other clause wherein said step of providing at least one inter-joint stability sensor comprises providing an inertial measurement unit.
292. A method of measuring inter-joint stability as described in clause 291 or any other clause wherein said step of providing an inertial measurement unit comprises providing a gyroscopic inertial measurement unit.
293. A method of measuring inter-joint stability as described in clause 291 or any other clause wherein said step of providing an inertial measurement unit comprises providing a magnetic inertial measurement unit.
294. A method of measuring inter-joint stability as described in clause 272 or any other clause further comprising the step of transmitting said at least one inter-joint stability sensor data.
295. A method of measuring inter-joint stability as described in clause 294 or any other clause wherein the step of transmitting said at least one inter-joint stability sensor data comprises transmitting said at least one inter-joint stability sensor data to an external application.
296. A method of measuring inter-joint stability as described in clause 295 or any other clause wherein said step of transmitting said at least one inter-joint stability sensor data to an external application comprises transmitting at least one inter-joint stability sensor data to a cellular phone application.
297. A method of measuring inter-joint stability as described in clause 295 or any other clause wherein said step of transmitting said at least one inter-joint stability sensor data to an external application comprises transmitting at least one inter-joint stability sensor data to a computer application.
298. A method of measuring inter-joint stability as described in clause 295 or any other clause wherein said step of transmitting said at least one inter-joint stability sensor data to an external application comprises transmitting at least one inter-joint stability sensor data to a cloud computing system.
299. A method of measuring inter-joint stability as described in clause 272 or any other clause further comprises the step of containing an inter-joint stability measurement system with a wearable article.
300. A method of measuring inter-joint stability as described in clause 299 or any other clause wherein said step of containing an inter-joint stability measurement system comprises containing said joint stability measurement system with a compression pant.
301. A method of measuring inter-joint stability as described in clause 299 or any other clause wherein said step of containing an inter-joint stability measurement system comprises containing said joint stability measurement system with a compression sleeve.
302. A method of measuring inter-joint stability as described in clause 299 or any other clause wherein said step of containing an inter-joint stability measurement system comprises containing said joint stability measurement system with a user-adjustable belt.
303. A method of measuring inter-joint stability as described in clause 299 or any other clause wherein said step of containing an inter-joint stability measurement system comprises containing said joint stability measurement system with a compression belt.
304. A method of measuring inter-joint stability as described in clause 299 or any other clause wherein said step of containing an inter-joint stability measurement system comprises containing said joint stability measurement system with a compression shirt.
305. A method of measuring inter-joint stability as described in clause 272 or any other clause further comprising a step of calculating joint range as a function of inter-joint acceleration.
306. A method of measuring inter-joint stability as described in clause 305 or any other clause wherein said step of calculating joint range as a function of inter-joint acceleration comprises calculating joint range as a function of inter-joint acceleration on an external application utilizing said at least one joint stability sensor data.
307. A method of measuring inter-joint stability as described in clause 305 or any other clause wherein said step of calculating joint range as a function of inter-joint acceleration on an external application utilizing said at least one inter-joint stability sensor data comprises transmitting at least one inter-joint stability sensor data to a cellular phone application.
308. A method of measuring inter-joint stability as described in clause 305 or any other clause wherein said step of calculating joint range as a function of joint acceleration and/or joint angular velocity on an external application utilizing said at least one inter-joint stability sensor data comprises transmitting at least one joint stability sensor data to a computer application.
309. A method of measuring inter-joint stability as described in clause 305 or any other clause wherein said step of calculating joint range as a function of joint acceleration and/or joint angular velocity on an external application utilizing said at least one inter-joint stability sensor data comprises transmitting at least one joint stability sensor data to a cloud computing system.
310. A method of measuring inter-joint stability as described in clause 272 or any other clause further comprising a step of actuating a feedback system in response to said step of measuring inter-joint acceleration and inter-joint angular velocity with said at least one inter-joint stability sensor.
311. A method of measuring inter-joint stability as described in clause 310 or any other clause wherein said step of actuating a feedback system in response to said step of measuring inter-joint acceleration and inter-joint angular velocity with said at least one inter-joint stability sensor comprises actuating a haptic feedback system.
312. A method of measuring inter-joint stability as described in clause 311 or any other clause wherein said step of actuating a haptic feedback system comprises actuating a vibrating actuator.
313. A method of measuring inter-joint stability as described in clause 311 or any other clause wherein said step of actuating a haptic feedback system comprises actuating an actuator that warms the skin of a user.
314. A method of measuring inter-joint stability as described in clause 310 or any other clause wherein said step of actuating a feedback system in response to said step of measuring inter-joint acceleration and inter-joint angular velocity with said at least one inter-joint stability sensor comprises utilizing a visual indicator.
315. A method of measuring inter-joint stability as described in clause 314 or any other clause wherein said step of utilizing a visual indicator comprises illuminating an at least one light emitting diode.
316. A method of measuring inter-joint stability as described in clause 130 or any other clause wherein said step of utilizing a visual indicator comprises displaying a notification on an external application.

As can be easily understood from the foregoing, the basic concepts of various embodiments, of the present invention(s) may be embodied in a variety of ways. It involves neuromuscular feedback techniques as well as devices to accomplish the appropriate neuromuscular feedback techniques. In this application, the neuromuscular feedback techniques are disclosed as part of the results shown to be achieved by the various devices described and as steps that are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this provisional application is intended to serve as a basic description. The reader should be aware that the specific discussion may not explicitly describe all embodiments, possible: many alternatives are implicit. It also may not fully explain the generic nature of the various embodiments, of the invention(s) and may not explicitly show how each feature or element can actually be representative of a broader function of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc. as well as terms that encompass largely but not wholly that which is specified. Further, for this application, if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a range, stability, or condition in a particular input, output, or at a particular stage can be specified as percentages that include 80%, 60%, 50%, 40%, and even 30% of the time the specified condition exists. Again, these are implicitly included in this disclosure and should (and, it is believed, would) be understood to a person of ordinary skill in this field. Where the application is described in device-oriented terminology, each element of the device implicitly performs a function. Apparatus claims may not only be included for the device described but also method or process claims may be included to address the functions of the embodiments, and that each element performs. Neither the description nor the terminology is intended to limit the scope of the claims that will be included in any subsequent patent application.

It should also be understood that a variety of changes may be made without departing from the essence of the various embodiments, of the invention(s). Such changes are also implicitly included in the description. They still fall within the scope of the various embodiments, of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the embodiments, of invention(s) both independently and as an overall system.

Further, each of the various elements of the embodiments, of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment, of any apparatus embodiment, a method or process embodiment, or even merely a variation of any element of these. Particularly, it should be understood that as the disclosure relates to elements of the various embodiments, of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments, of the invention(s) is entitled. As but one example, it should be understood that all actions may be expressed as a means for taking that action or as an element that causes that action. Similarly, each physical element disclosed should be understood to encompass a disclosure of the action which that physical element facilitates. Regarding this last aspect, as but one example, the disclosure of a “sensor” should be understood to encompass disclosure of the act of “sensing” or “providing a signal”—whether explicitly discussed or not—and, conversely, were there effectively disclosure of the act of “sensing,” such a disclosure should be understood to encompass disclosure of a “sensor” and even a “means for sensing.”) Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components that are configured to, or configured and arranged to, achieve a particular result, use, purpose, situation, function, or operation, or as components that are capable of achieving a particular result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The Provisional Patent Application or other information statement filed with the application are hereby appended and hereby incorporated by reference, however, as to each of the above, to the extent that such information or statements incorporated by reference might be considered inconsistent with the patenting of the various embodiments, of invention(s) such statements are expressly not to be considered as made by the applicant(s).

### REFERENCES TO BE INCORPORATED BY REFERENCE

### US Patents

| | Kind | | |
|---|---|---|---|
| Pat. No. | Code | Date Issued | Patentee |
| 1,922,763 | | 1993 Aug. 15 | Gricks, Rudolph |
| 4,636,206 | | 1987 Jan. 13 | Ederati, Richard M., et al. |
| 5,338,315 | | 1994 Aug. 16 | Baker, Freddie R. |
| 9,907,689 | B2 | 2018 Mar. 6 | Persichina, Michael Thomas, |
| | | | et al. |
| 10,258,495 | B2 | 2019 Apr. 16 | Luce, Donna E |

### US Publications

| Publication No. | Kind Code | Date Published | Patentee |
|---|---|---|---|
| 2013/0207889 | A1 | 2013 Aug. 15 | Lumo Bodytech, Inc. |

### Foreign Patents

| | Kind | Country | Date | |
|---|---|---|---|---|
| Pat. No. | Code | Code | Published | Patentee |
| 2017/120367 | A1 | WO | 2017 Jul. 13 | Wearable |
| | | | | Experiments Inc. |

### Non-Patent Literature

J Cholewicki et al., Mechanical stability of the in vivo lumbar spine: implications for
injury and chronic low back pain. Clinical Biomechanics Vol. 11, No. 1, pp. 1-15, 1996.
Elio Locatelli, The importance of anaerobic glycolysis and stiffness in the sprints (60,
100 and 200 metres). New Studies in Athletics, No. 2-3/1996. 5 pages.
Nigel Joseph et al., Examining Core Dysfunction in Football Athletes through
Interdisciplinary Systemic Design Problem Solving. Cleveland, OH 2018. 2 pages.
Anthony G. Schache et al., Relation of Anterior pelvic tilt during running to clinical and
kinematic measures of hip extension. Br J Sports Med 2000; 34: 279-283. Feb. 29, 2000.
Thomas W. Nesser, The Relationship between core strength and performance in
division I female soccer players. Journal of Exercise Physiology, Vol. 12, No. 2,
Apr. 4, 2009. 8 pages.

Thus, the applicant(s) should be understood to have support to claim and make claims to embodiments, including at least: i) each of the neuromuscular feedback wearable, haptic feedback, and joint positioning devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, it should be understood that in characterizing these and all other aspects of the various embodiments, of the invention(s)—whether characterized as a device, a capability, an element, or otherwise, because all of these can be implemented via software, hardware, or even firmware structures as set up for a general-purpose computer, a programmed chip or chipset, an ASIC, application-specific controller, subroutine, or other known programmable or circuit-specific structure—it should be understood that all such aspects are at least defined by structures including, a person of ordinary skill in the art would well recognize: hardware circuitry, firmware, programmed application-specific components, and even a general-purpose computer programmed to accomplish the identified aspect. For such items implemented by programmable features, the applicant(s) should be understood to have support to claim and make a statement of invention(s) to at least: xv) processes performed with the aid of or on a computer, machine, or computing machine as described throughout the above discussion, xvi) a programmable apparatus as described throughout the above discussion, xvii) a computer-readable memory encoded with data to direct a computer comprising means or elements which function as described throughout the above discussion, xviii) a computer, machine, or computing machine configured as herein disclosed and described, xix) individual or combined subroutines and programs as herein disclosed and described, xx) a carrier medium carrying computer-readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) a processor executing instructions that act to achieve the steps and activities detailed, xxvi) circuitry configurations (including configurations of transistors, gates, and the like) that act to sequence and/or cause actions as detailed, xxvii) computer-readable medium(s) storing instructions to execute the steps and cause activities detailed, xxviii) the related methods disclosed and described, xxix) similar, equivalent, and even implicit variations of each of these systems and methods, XXX) those alternative designs which accomplish each of the functions shown as are disclosed and described, xxxi) those alternative designs and methods which accomplish each of the functions shown as are implicit to accomplish that which is disclosed and described, xxxii) each feature, component, and step shown as separate and independent inventions, and xxxiii) the various combinations of each of the above and of any aspect, all without limiting other aspects in addition.

In addition, the applicant(s) should be understood to have support to claim and make a statement of the invention(s) that may include claims directed to:
providing desired haptic feedback;
systems to achieve desired haptic feedback;
a haptic feedback device;
specific configurations of haptic feedback devices;
components or structures for haptic feedback device;
systems that enable a manufacturer or other user to customize haptic feedback devices;
specific configurations of efficient haptic feedback;
systems that enable optimized haptic feedback;
systems to metrically control haptic feedback;
systems to utilize complex ratios in haptic feedback;
components or structures to utilize complex ratios in haptic feedback;
providing desired joint determinations;
systems to achieve desired joint determinations;
a joint determination device;
specific configurations of joint determinations devices;
components or structures for joint determinations device;
a neuromuscular feedback device;
specific configurations of neuromuscular feedback devices;
components or structures for neuromuscular feedback device;
systems that enable a manufacturer or other user to customize neuromuscular feedback devices;
specific configurations of efficient neuromuscular feedback;
systems that enable optimized neuromuscular feedback;
systems to metrically control neuromuscular feedback;
systems to utilize complex ratios in neuromuscular feedback;
components or structures to utilize complex ratios in neuromuscular feedback; and or
any permutation or combination of any of the above or any elements of the above.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be revisited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities: one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments, of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A joint movement wellbeing system comprising:
a mobile power system;
a joint movement sensor;
a microcontroller configured and arranged to determine joint range information as a function of joint acceleration, joint angular velocity, or both;
a joint stability processor;
a joint stability indicator; and
a preferred joint range information-based indicator.

2. A joint movement wellbeing system as described in claim 1 wherein said microcontroller comprises a real-time joint range processor.

3. A joint movement wellbeing system as described in claim 1 wherein said processor comprises a real-time joint stability processor.

4. A joint movement wellbeing system as described in claim 1 wherein said joint stability processor comprises a joint stability processor configured and arranged to assess joint stability using aspects selected from:
joint optimal movement aspects,
user baseline aspects, and
consideration of at least one contemporaneously sensed joint movement,
muscle activation,
joint muscle activation, and
core muscle activation.

5. A joint movement wellbeing system as described in claim 1 wherein said microcontroller comprises a microcontroller configured and arranged to assess joint range using aspects selected from:
joint acceleration,
joint angular velocity,
joint stability,
muscle activation,
joint muscle activation, and
core muscle activation.

6. A joint movement wellbeing system as described in claim 1 wherein said mobile power system comprises a battery, a capacitor, or a piezo-electric power generator.

7.-8. (canceled)

9. A joint movement wellbeing system as described in claim 1 wherein said joint movement sensor comprises an accelerometer, a joint-segment sensor, or a joint-region sensor.

10. A joint movement wellbeing system as described in claim 1 wherein said joint movement sensor comprises an accelerometer configured and arranged to measure non-optimal joint movement, configured and arranged to measure core muscle activation, or configured and arranged to measure joint stability as a function of supporting muscle activation.

11. A joint movement wellbeing system as described in claim 1 wherein said joint movement sensor comprises a sensor system configured and arranged to measure joint movement or joint angular velocity in three different dimensions.

12.-14. (canceled)

15. A joint movement wellbeing system as described in claim 1 wherein said joint comprises a joint-segment or a sacroiliac joint.

16. (canceled)

17. A joint movement wellbeing system as described in claim 1 wherein said joint comprises a ball-and-socket joint or a hinge joint.

18. (canceled)

19. A joint movement wellbeing system as described in claim 1 wherein said joint comprises a condyloid joint or a pivot joint.

20. (canceled)

21. A joint movement wellbeing system as described in claim 1 wherein said joint comprises a gliding joint or a saddle joint.

22.-24. (canceled)

25. A joint movement wellbeing system as described in claim 1 further comprising a transmitter configured and arranged to transmit data to an external application.

26.-29. (canceled)

30. A joint movement wellbeing system as described in claim 1 further comprising a wearable containment system configured and arranged to contain said joint range measurement system.

31. A joint movement wellbeing system as described in claim 1 wherein said microcontroller is configured and arranged to utilize one of a duration of an in-preferred joint range and a duration of an out-of-preferred joint range.

32. (canceled)

33. A joint movement wellbeing system as described in claim 1 wherein said joint stability processor is configured and arranged to utilize one of a duration of an unstable joint determination and a duration of a stable joint determination.

34. (canceled)

35. A joint range feedback system comprising:
a mobile power system;
a joint movement sensor;
a joint movement range processor; and —a user feedback signal.

36. A method of considering joint wellbeing comprising the steps of:
portably powering a joint movement sensing system;
sensing joint movement by said at least one joint movement sensor system; —determining preferred joint range information as a function of joint acceleration, joint angular velocity, or both; and
indicating information based on said preferred joint range.

37. A method of considering joint wellbeing comprising the steps of:
portably powering a joint movement sensing system;
sensing joint movement by said at least one joint movement sensor system;
determining joint stability; and
indicating joint stability.
